(19) United States
(12) Patent Application Publication
(10) Pub. No.: US 2026/0001881 A1
(43) Pub. Date: Jan. 1, 2026

(54) COMPOUNDS AND METHODS FOR MODULATION OF G-PROTEIN-COUPLED RECEPTORS

(71) Applicant: PRIMETIME LIFE SCIENCES, LLC, Germantown, MD (US)

(72) Inventor: Janak Khimchand PADIA, Germantown, MD (US)

(21) Appl. No.: 17/055,591

(22) PCT Filed: Jul. 26, 2019
(86) PCT No.: PCT/US2019/043558

§ 371 Date: Nov. 15, 2020

Related U.S. Application Data
(60) Provisional application No. 62/676,938, filed on May 26, 2018.

(51) Int. Cl. C07D 473/34; A61K 31/52; A61K 31/7076; C07D 473/40; C07D 519/00; C07H 19/167

(52) U.S. Cl. CPC C07D 473/34; A61K 31/52; A61K 31/7076; C07D 473/40; C07D 519/00; C07H 19/167

(57) ABSTRACT

The present invention relates to compounds that modulate G-Protein-Coupled Receptors, to process of preparing these compounds, their salts, prodrugs, and metabolites, to pharmaceutical compositions containing these compounds, and to methods of using these compounds for treating a wide variety of medical conditions, diseases or disorders.

### GOVERNMENT SUPPORT CLAUSE

This invention was made with government support under R44 MH116741 awarded by the National Institutes of Health. The government has certain rights in the invention.

### FIELD OF THE INVENTION

The present invention relates to compounds that modulate G-Protein-Coupled Receptors, to process of preparing these compounds, their salts, prodrugs, and metabolites, to pharmaceutical compositions containing these compounds, and to methods of using these compounds for treating a wide variety of medical conditions, diseases or disorders.

### BACKGROUND OF THE INVENTION

G-protein-coupled receptors (GPCRs) are the largest family of receptors in many organisms, including worms, rodents, and humans. GPCRs are seven-transmembrane proteins that are activated by a ligand transduce that information to the inside of the cell through conformational changes. The conformational changes activate heterotrimeric G-proteins, which execute the downstream signaling pathways through the recruitment and activation of cellular enzymes. The highly specific ligand-GPCR interaction prompts an efficient cellular response, which is vital for the health of the cell and organism.

GPCRs are involved in nearly every aspect of animal life, from early development and heart function to neuronal activity. Mutations in GPCRs are linked to several human diseases. Cell migration is another process that requires GPCRs, in both beneficial and detrimental ways. GPCRs are a major target in pharmacology because of their mediation in different functions within central and peripheral nervous systems. More than 40% of all medicines available today act on a GPCR for treatment of a variety of diseases. The GPCRs are divided in five into classes: Class A (rhodopsin-like), Class B (secretin receptor family), Class C (glutamate), adhesion family and frizzled family (Schiöth, et al., General and comparative endocrinology, 142, 94-101, (2005)). GPCRs are involved in a wide variety of physiological processes including visual, gustatory, and smell senses and regulation of the immune system, inflammation, cancer, neurodegenerative, and cerebrovascular diseases.

Some of the key GPCRs are 5-hydroxytryptamine receptors, acetylcholine receptors (muscarinic), adenosine receptors, adhesion class GPCRs, adrenoceptors, angiotensin receptors, apelin receptor, bile acid receptor, bombesin receptors, bradykinin receptors, calcitonin receptors, calcium-sensing receptors, cannabinoid receptors, chemerin receptor, chemokine receptors, cholecystokinin receptors, complement peptide receptors, corticotropin-releasing factor receptors, dopamine receptors, endothelin receptors, estrogen (G protein-coupled) receptor, formylpeptide receptors, free fatty acid receptors, frizzled class GPCRs, GABAB receptors, galanin receptors, ghrelin receptor, glucagon receptor family, glycoprotein hormone receptors, gonadotrophin-releasing hormone receptors, GPR18, GPR55 and GPR119, histamine receptors, hydroxycarboxylic acid receptors, kisspeptin receptors, leukotriene, lipoxin and oxoeicosanoid receptors, lysophospholipid (LPA) receptors, lysophospholipid (S1P) receptors, melanin-concentrating hormone receptors, melanocortin receptors, melatonin receptors, metabotropic glutamate receptors, motilin receptor, neuromedin u receptors, neuropeptide FF/neuropeptide AF receptors, neuropeptide S receptor, neuropeptide W/neuropeptide B receptors, neuropeptide Y receptors, neurotensin receptors, opioid receptors, orexin receptors, oxoglutarate receptor, P2Y receptors, parathyroid hormone receptors, peptide P518 receptor, platelet-activating factor receptor, prokineticin receptors, prolactin-releasing peptide receptor, prostanoid receptors, proteinase-activated receptors, relaxin family peptide receptors, somatostatin receptors, succinate receptor, tachykinin receptors, thyrotropin-releasing hormone receptors, trace amine receptor, urotensin receptor, vasopressin and oxytocin receptors, VIP and PACAP receptors.

Purine is the most widely distributed heterocycle in nature, and many purine derivatives, adenine, and guanine or their respective nucleoside/nucleotide derivatives are the most common class of nitrogen heterocycles which play crucial roles in a wide variety of functions of living species. The analogs of purines have found utility both as chemotherapeutics (antiviral, antibiotic, and anticancer agents) and pharmacodynamic entities (regulation of myocardial oxygen consumption and cardiac blood flow). They can also act as substrates or inhibitors of enzymes of purine metabolism (ADA, Guanase, HGPRTase, PNPase, etc.) to exert their chemotherapeutic property. Also, their ability to act as agonists or antagonists of GPCRs is the basis for modulation of pharmacodynamic property in several therapeutics.

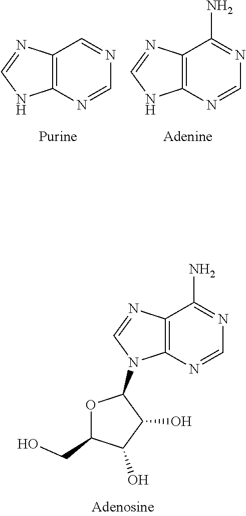

Adenine is a purine derivative with amino substitution on the 6-position whereas adenosine is further derivatization of adenine with substitution of ribose sugar molecule on the N-9 position.

Adenosine is an endogenous purine nucleoside, and it modulates many physiological processes. Cellular signaling by adenosine occurs through four known adenosine receptor (AR) subtypes (A1, A2A, A2B, and A3). ARs play an important role in physiology and pathophysiology and therefore represent attractive drug targets for a range of conditions. Cells of the immune system express these receptors and are responsive to the modulatory effects of adenosine in an inflammatory environment. Animal models of asthma, ischemia, arthritis, sepsis, inflammatory bowel disease, and wound healing have helped to elucidate the regulatory roles of the various adenosine receptors in dictating the development and progression of the disease. Modulation of adenosine receptors have many therapeutic effects in cardiovascular disease (for example: arrhythmia (Zablocki, et al., Current topics in medicinal chemistry, 4, 839-854, (2004); Fraser, et al., Journal of Pharmacology and Experimental Therapeutics, 305, 225-231, (2003); Bayes, et al., Methods and findings in experimental and clinical pharmacology, 25, 831-55, (2003)), ischaemia (Shneyvays, et al., Cell calcium, 36, 387-396, (2004); Tracey, et al., Cardiovascular research, 40, 138-145, (1998); Mozzicato, et al., The FASEB journal, 18, 406-408, (2004); Schindler, et al., British journal of pharmacology, 144, 642-650, (2005)), vasodilation (Schindler, et al., British journal of pharmacology, 144, 642-650, (2005); Varani, et al., Circulation, 102, 285-289, (2000))), nervous system disorders (for example: dementia and anxiety disorders (Maemoto, et al., Journal of pharmacological sciences, 96, 42-52, (2004)), pain (Sawynok, European journal of pharmacology, 347, 1-11, (1998); Johansson, et al., Proceedings of the National Academy of Sciences, 98, 9407-9412, (2001); Wu, et al., Pain, 113, 395-404, (2005)), Parkinson's disease (Xu, et al., Pharmacology & therapeutics, 105, 267-310, (2005); Hauser, et al., Neurology, 61, 297-303, (2003); Chen, et al., Journal of Neuroscience, 19, 9192-9200, (1999)), Ischaemia and neuroprotection (Yu, et al., Nature medicine, 10, 1081, (2004); Olsson, et al., European Journal of Neuroscience, 20, 1197-1204, (2004); Turner, et al., Proceedings of the National Academy of Sciences, 100, 11718-11722, (2003)), Sleep (Porkka-Heiskanen, et al., Science, 276, 1265-1268, (1997); Stenberg, et al., Journal of sleep research, 12, 283-290, (2003); Basheer, et al., Progress in neurobiology, 73, 379-396, (2004)), renal system disorders (Pingle, et al., Journal of Biological Chemistry, 279, 43157-43167, (2004); Gottlieb, et al., Circulation, 105, 1348-1353, (2002); Lee, et al., American Journal of Physiology-Renal Physiology, 286, F298-F306, (2004)), pulmonary disorders (Sun, et al., The Journal of clinical investigation, 115, 35-43, (2005); Holgate, British journal of pharmacology, 145, 1009-1015, (2005); Fozard, et al., European journal of pharmacology, 438, 183-188, (2002)), inflammatory disorders (Sitkovsky, et al., Annu. Rev. Immunol., 22, 657-682, (2004); Ohta, et al., Nature, 414, 916, (2001)), endocrine disorders (Dong, et al., Diabetes, Obesity and Metabolism, 3, 360-366, (2001); Harada, et al., Journal of medicinal chemistry, 44, 170-179, (2001)), cancer (Antonioli, et al., Nature Reviews Cancer, 13, 842, (2013); Adamson, et al., Pharmacology, 15, 84-89, (1977); Stagg, et al., Oncogene, 29, 5346, (2010)), visual disorders (Okamura, et al., Bioorganic & medicinal chemistry letters, 14, 3775-3779, (2004); Avila, et al., Investigative ophthalmology & visual science, 43, 3021-3026, (2002)).

Adenosine itself has been modified to generate ligands for ARs, and extensive structure-activity relationship (SAR) studies have probed. Of the major modifications, analogs with the N6 or 2-position of the adenine moiety and in the 3′-, 4′- or 5′-position of the ribose moiety have produced valuable agonist and antagonists for ARs. Crystal structure of the seven-transmembrane protein rhodopsin, supported by mutagenesis studies, has provided good understanding of ligand recognition and insights into conformational dynamics. With these information, rational drug design and molecular modeling using have been used for design and produced potent and selective ligands of ARs (Kim, et al., Journal of medicinal chemistry, 46, 4847-4859, (2003); Tchilibon, et al., Journal of medicinal chemistry, 48, 1745-1758, (2005); Moro, et al., Current drug discovery technologies, 2, 13-21, (2005); Moro, et al., Medicinal research reviews, 26, 131-159, (2006); Gao, et al., Journal of medicinal chemistry, 45, 4471-4484, (2002)). Adenosine derivatives have also shown antagonist activities for 5HT_{2B}R and 5HT_{2C}R (Tosh, et al., Journal of medicinal chemistry, 59, 11006-11026, (2016)).

Several GPCR ligands have entered clinical trials; however, problems with high lipophilicity and corresponding low water solubility and bioavailability have limited their clinical development (Hess, et al., Journal of medicinal chemistry, 43, 4636-4646, (2000)). Compound A and Compound B have shown remarkable activities and selectivity for Air agonistic and A3R agonistic activities, respectively.

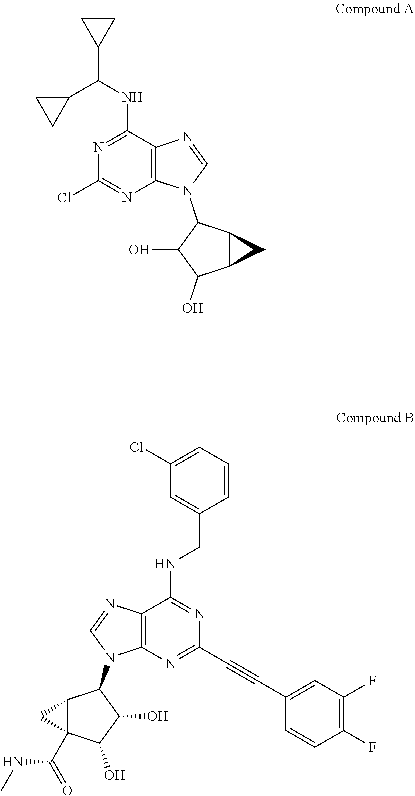

### SUMMARY OF THE INVENTION

The present invention relates to compounds that modulate G-Protein-Coupled Receptors, to process of preparing these compounds, their salts, prodrugs, and metabolites, to pharmaceutical compositions containing these compounds, and to methods of using these compounds for treating a wide variety of medical conditions, diseases or disorders. In one aspect, the invention provides compounds of Formula IA and Formula IB, or its pharmaceutically acceptable salt or a prodrug or metabolite(s) thereof

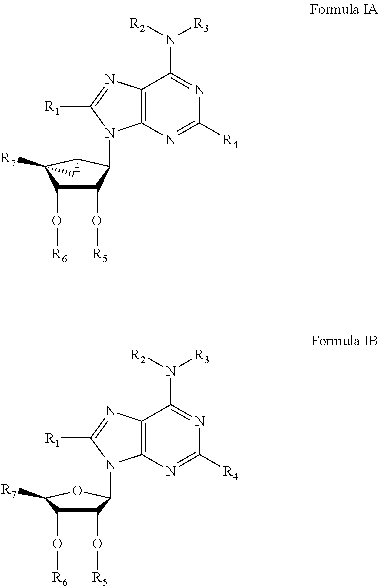

wherein
R₁ is selected from hydrogen, alkyl, alkenyl, alkynyl, aryl, heteroaryl wherein said alkyl, alkenyl, alkynyl, aryl, heteroaryl each are optionally substituted with one or more R₈;
R₂ is selected from hydrogen, amino acid, alkyl, alkenyl, alkynyl, heteroalkyl, aryl, arylalkyl, heteroaryl, heteroarylalkyl, cycloalkyl and heterocycloalkyl wherein said amino acid, alkyl, alkenyl, alkynyl, heteroalkyl, aryl, arylalkyl, heteroaryl, heteroarylalkyl, cycloalkyl and heterocycloalkyl each are optionally substituted with one or more R₈;
R₃ is selected from hydrogen, alkyl, aryl, arylalkyl, heteroaryl, heteroarylalkyl,

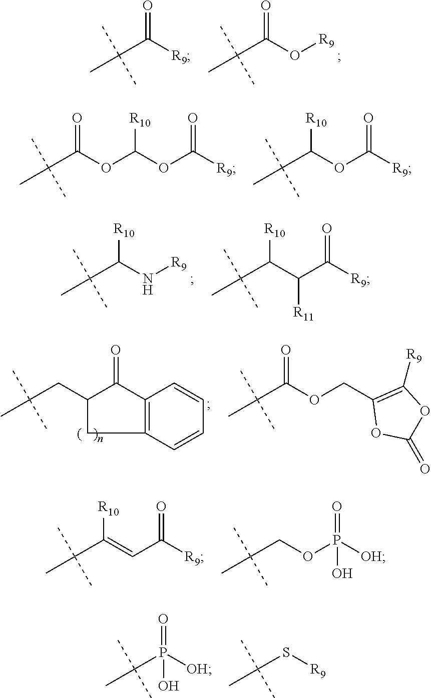

wherein n=1, 2, or 3;
R₄ is selected from hydrogen, amino acid, halogen, alkylthio, alkyl, alkenyl, alkynyl, heteroalkyl, aryl, arylalkyl, heteroaryl, heteroarylalkyl, cycloalkyl and heterocycloalkyl wherein said amino acid, alkyl, alkenyl, alkynyl, heteroalkyl, aryl, arylalkyl, heteroaryl, heteroarylalkyl, cycloalkyl and heterocycloalkyl each are optionally substituted with one or more R₈;
R₅ and R₆ are independently selected from hydrogen, alkyl,

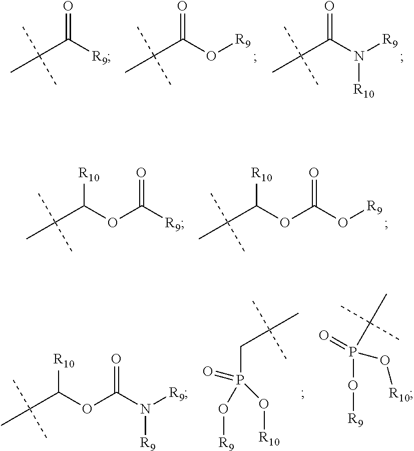

and R₅ and R₆ can be connected to form any of the following cyclic ring systems

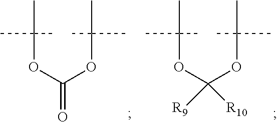

wherein R₉ and R₁₀ are selected from hydrogen, alkyl, heteroalkyl, aryl, arylalkyl, heteroaryl, heteroarylalkyl, cycloalkyl and heterocycloalkyl wherein said each are optionally substituted with one or more R₈;
and R₉ and R₁₀ can be connected to form a cycloalkyl or heterocycloalkyl include one or more nitrogen (N), oxygen (O) or sulfur (S) atoms; wherein said cycloalkyl, heterocycloalkyl are optionally substituted with one or more R₈;
R₇ is selected from hydrogen, alkyl

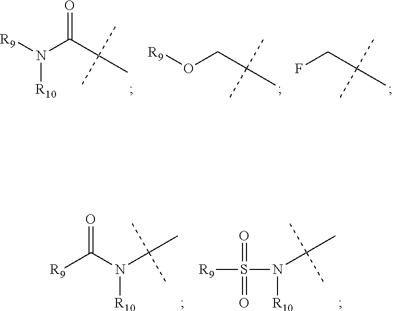

R₈ is independently selected for each occurrence from the group consisting of hydrogen, amino acid, lower alkyl, lower alkenyl, lower alkynyl, cycloalkyl, heterocycloalkyl aryl, heteroaryl, alicyclic, arylalkyl, heteroarylalkyl, lower alkoxy, aryloxy, amino, alkylamino, dialkylamino, diarylalkylamino, alkylthio, arylthio, heteroarylthio, oxo, oxa, —C(O)—R₁₂, —C(O)OR₁₂, —C(O)NR₁₂R₁₃, —C(S)NR₁₂R₁₃, CO₂H, acyloxy, halo, —CN, —NO₂, —N₃, —SH, —OH, —CH(CF₃)NR₁₂R₁₃, —C(═N)NR₁₂, perhaloalkyl, perhaloalkoxy, perhaloacyl, guanidinyl, pyridinyl, thiophene, furanyl, indolyl, indazolyl, phosphonate, phosphonic acid, phosphate, phosphoramide, sulfonate, sulfone, sulfate, sulphonamide, carbamate, urea, thiourea, thioamide and thioalkyl; wherein said heterocycloalkyl or heteroaryl include one or more nitrogen (N), oxygen (O) or sulfur (S) atoms; wherein R₁₂ and R₁₃ are independently selected from H, methyl, ethyl, and benzyl;
R₉, R₁₀, and R₁₁ are each independently selected from hydrogen, amino acid, lower alkyl, lower alkenyl, lower alkynyl, cycloalkyl, heterocycloalkyl aryl, heteroaryl, alicyclic, arylalkyl, heteroarylalkyl, lower alkoxy, aryloxy, —NO₂, —N₃, —SH, or —OH, each are optionally substituted with one or more R₈.

According to one embodiment, a pharmaceutical composition comprising a compound of Formula IA or Formula IB or a pharmaceutically acceptable salt, ester or prodrug or metabolite thereof in association with a pharmaceutically acceptable diluent or carrier.

According to one embodiment, a pharmaceutical composition comprising a compound of Formula IA or Formula IB or a pharmaceutically acceptable salt, ester or prodrug or metabolite thereof in association with a pharmaceutically acceptable diluent or carrier to form a formulation system for delivering the compound.

According to one embodiment, a pharmaceutical composition comprising a compound of Formula IA or Formula IB as defined in claim 1 or a pharmaceutically acceptable salt, ester or prodrug or metabolite thereof in a combination of other pharmaceutically active agent(s).

According to one embodiment, a compound of Formula IA or Formula IB or a pharmaceutically acceptable salt, ester or prodrug or metabolite thereof for use in therapy.

According to one embodiment, the use of a compound of Formula IA or Formula IB or a pharmaceutically acceptable salt, ester or prodrug or metabolite thereof in the preparation of a medicament for the treatment of cardiovascular disease, nervous system disorders, Parkinson's disease, neurodegenerative disorders, ischaemia and neuroprotection, Sleep, renal system disorders, pulmonary disorders, inflammatory disorders, endocrine disorders, cancer, visual disorders.

According to one embodiment, a process of producing a compound of Formula IA or Formula IB or its pharmaceutically acceptable salt or prodrug or metabolite.

According to another aspect, a method for the treatment or prevention of a disease or condition modulated by GPCR(s) is provided and includes the step of administering a compound as provided herein. Any of the methods or uses provided herein may include administering to a subject a therapeutically effective amount of a compound as provided herein, including salt or polymorph thereof, or a pharmaceutical composition that includes such compounds.

The invention relates to formulation systems loaded with compounds of this invention mention above which said systems have improved biopharmaceutical properties for solubility, drug concentration in target tissue (s), in vivo efficacy and safety, improved quality (fineness and homogeneity of the particles, drug inclusion) and improved physical stability of the particulate formulation (no aggregation or gel formation). The compound of this invention can be appropriately formulated for desired delivery systems such as oral drug delivery (immediate release, delayed-release, prolonged-release, modified release), parenteral drug delivery, ophthalmic drug delivery, nasal drug delivery, rectal drug delivery, colon-specific drug delivery, topical drug delivery, and CNS or brain drug delivery by powder injection; or by buccal, sublingual, or intranasal absorption. Pharmaceutical compositions may be formulated in unit dose form, or in multiple or subunit doses.

The manner in which the compounds or their pharmaceutical composition set forth herein may be administered can vary. According to one embodiment, the compounds can be administered orally. Preferred pharmaceutical compositions may be formulated for oral administration in the form of tablets, capsules, caplets, syrups, solutions, and suspensions. Such oral formulations can be provided in modified release dosage forms such as time-release tablet and capsule formulations. Pharmaceutical compositions can also be administered via injection, namely, intravenously, intramuscularly, subcutaneously, intraperitoneally, intra-arterial, intrathecally, and intracerebroventricularly.

Suitable carriers for injection are well known to those of skill in the art and include 5% dextrose solutions, saline, and phosphate buffered saline.

According to another aspect, a formulation of the compound of present invention can be prepared by entrapping the compound in liposomes, or albumin. The formulation of the compound of present invention can be prepared by using nanoparticles, nanocapsules or nanospheres using appropriate excipient(s) such as cyclodextrins, mannitol, sodium dodecyl sulfate, albumin, polysorbate 80, trehalose, sucrose, lactose, tromethamine, sodium chloride, gelatin, amino acids.

In another embodiment of the invention, stabilizing excipients for preparing formulations of compound of Formula IA and Formula IB are selected from hydrophobicity inducing agents.

These agents may be represented by magnesium Stearate, Stearic acid, glyceryl Stearate, glyceryl palmitostearate, Stearoyl macrogolglycerides, lauroyl macrogolglycerides, waxes and hydrogenated vegetable oils, among others.

The stabilizers may be included into the formulations for compound of Formula IA and Formula IB for the of the current invention in the amount Such that, for an individual stabilizer, the ratio of the parts by weight of stabilizer to parts by weight of the drug substance is from 0.1:1 to 50:1, preferably from 0.25:1 to 40:1; most preferably from 0.4:1 to 25:1. Combinations of stabilizing excipients may be used in all embodiments of the instant invention and may provide synergistic stabilizing action.

Stabilizers may be incorporated into formulations of a compound of Formula IA and Formula IB in a variety of ways. They may be intermixed with the drug Substance and/or other excipients or may be provided in the form of a coating on the compound of Formula IA and Formula IB-containing substrate. Water-based acidifiers may be used in the preparation of the formulations of the current invention as long as care is taken to eliminate or reduce water during the processing. Alternatively, excipients, such as bulking agents, may be pre-treated by the stabilizers prior to their incorporation into the formulation. Stabilization of compound of Formula IA and Formula IB may be also achieved by coating drug layered Substrates with coating polymers dissolved or dispersed in acidic Solution. These and further ways of using stabilizers are disclosed in more detail in the examples below. Additional excipients that can be used alone or in combination to formulate stable compound of Formula IA and Formula IB drug products in accordance with the current invention include bulking agents. Such as lactose anhydrous or lactose monohydrate, (i.e., Supertab 21AN, Ludipress, Ludipress LCE, Fast Flo Lactose, Supertose, Pharmatose, Respitose), glyceryl behenate, hypromel lose, ascorbic acid, benzoic acid, carbomer, low moisture microcrystalline cellulose (Avicel® grades PH-103, PH-112, PH-113, PH-200), colloidal silicon dioxide, dextrose (anhydrous), dextrose (anhydrous), maltol, fructose, glyceryl palmitostearate, glyceryl monostearate, guar gum, lactitol (anhydrous), magnesium carbonate, maltitol, maltose, mannitol, polyethylene oxide, Sorbitol. Sucrose, compressible Sugar, confectioner's Sugar, Xylitol; glidants such as talc, starch and colloidal silicon dioxide and the metallic Stearates; lubricants selected from talc, sodium Stearyl fumarate, hydrogenated vegetable oils, glyceryl palmitostearate, glyceryl behenate, poloxamer, Stearic acid, Stearyl alcohol, cetyl alcohol, waxes, and the metallic Stearates; wetting and solubility enhancing agents, such as sodium lauryl Sulfate, polyethylene glycol, PEG glyceryl esters, lecithin, poloxamer, the polysorbates, the polyoxyethylene alkyl ethers, polyethylene castor oil derivatives, polyethylene Stearate, and the Sorbitan esters. Through the use of stabilizers and low levels of moisture as described above, the inventors were able to realize one goal of the current invention: to provide stable IR formulations of a compound of Formula IA and Formula IB that comprise not more than 5% of water. In yet further embodiment, the invention discloses stable IR formulations of a compound of Formula IA and Formula IB comprising stabilizing excipients. A further goal of the current invention is to utilize stabilization techniques described herein to provide stable MR formulations of a compound of Formula IA and Formula IB comprising active compound, at least one release controlling polymer that may be a non-pH-dependent polymer or a pH-dependent, enteric polymer, and at least one pharmaceutically acceptable excipient.

Further, the invention provides MR formulations of a compound of Formula IA and Formula IB comprising a compound of Formula IA and Formula IB, at least one release controlling polymer and at least one pharmaceutically acceptable excipient, wherein the total amount of residual water in the formulation is not more than 5% by weight of the formulation. The MR formulations of a compound of Formula IA and Formula IB exhibiting XR profile, or combination of XR and DR profile, or any combination of those with IR profile are disclosed herein. These specific release profiles are achieved by formulating compound of Formula IA and Formula IB, at least one release controlling polymer and one or more excipient in a variety of inventive formulations. The release controlling polymers of the current invention may be selected from non-pH-dependent polymers such as hydrophilic rate controlling compound that can be used to formulate MR multi-particulates or matrix tablets drug products, and hydrophobic rate-controlling compounds that exhibit limited or no water solubility; or enteric polymers that exhibit pH-dependent solubility.

Osmotic tablets can be formulated as a single or as a multiple layer core. In one embodiment, the osmotic tablet comprises a bilayer core, wherein one layer comprises agents to modulate drug release, such as a solubilizer, that are released in a Sustained manner, and the second layer comprises the drug and potentially other agents to modulate drug release. Stabilizers listed above may be contained in at least one layer of the osmotic formulation. An overcoat of drug can be applied to the osmotic tablet following a functional coating to provide an immediate release component to the dosage form. Alternatively, the osmotic tablet may be coated with an enteric polymer on top of the semipermeable rate-controlling membrane providing a DR/XR profile.

Pharmaceutical compositions may also be administered using other means, for example, rectal administration. Formulations useful for rectal administration, such as suppositories, are well known to those of skill in the art. The compounds can also be administered by inhalation, for example, in the form of an aerosol; topically, such as, in lotion form; transdermally, such as, using a transdermal patch (for example, by using technology that is commercially available from Novartis and Alza Corporation); by powder injection; or by buccal, sublingual, or intranasal absorption. Pharmaceutical compositions may be formulated in unit dose form, or in multiple or subunit doses.

The administration of the pharmaceutical compositions described herein can be intermittent, or at a gradual, continuous, constant, or controlled rate. The pharmaceutical compositions may be administered to a warm-blooded animal, for example, a mammal such as a human being. In addition, the time of day and the number of times per day that the pharmaceutical composition is administered can vary.

The compounds, as provided herein may also be used for the preparation of a medicament for the treatment or prevention of a disease or condition modulated by GPCR(s). Methods for treating, preventing, delaying the onset of, or slowing the progression of disorders mediated by GPCR(s) involved in the regulation or dysregulation of gene expression, in mammals in need of such treatment are also provided. The methods involve administering to a subject a therapeutically effective amount of a compound as provided herein, including a salt thereof, or a pharmaceutical composition that includes such compounds.

According to one embodiment, the methods for treating, preventing, delaying the onset of, or slowing the progression of disorders mediated by acetylated proteins involved in the regulation or dysregulation of gene expression, in mammals in need of such treatment include the administration of at least one compound as provided herein including, but not limited to, the compounds provided according to Formula I.

The compounds alone or in a pharmaceutical composition as provided herein may be used in the treatment of a variety of disorders and conditions and, as such, may be used in combination with a variety of other suitable therapeutic agents useful in the treatment or prophylaxis of those disorders or conditions. Thus, one embodiment of the present disclosure includes the administration of the compound of the present disclosure in combination with other therapeutic compounds. Such a combination of pharmaceutically active agents may be administered together or separately and, when administered separately, the administration may occur simultaneously or sequentially, in any order. The amounts of the compounds or agents and the relative timings of administration will be selected in order to achieve the desired therapeutic effect. The administration in a combination of a compound of the present disclosure with other treatment agents may be in combination by administration concomitantly in: (1) a unitary pharmaceutical composition including two or more compounds; or (2) separate pharmaceutical compositions each including one of the compounds. Alternatively, the combination may be administered separately in a sequential manner wherein one treatment agent is administered first and the other second. Such sequential administration may be close in time or remote in time.

Another aspect of the present disclosure includes combination therapy comprising administering to the subject a therapeutically or prophylactically effective amount of the compound of the present disclosure and one or more other therapy including chemotherapy, radiation therapy, gene therapy, or immunotherapy.

### DETAILED DESCRIPTION OF THE INVENTION

The present invention relates to compounds that modulate G-Protein-Coupled Receptors, to process of preparing these compounds, their salts, prodrugs, and metabolites, to pharmaceutical compositions containing these compounds, and to methods of using these compounds for treating a wide variety of medical conditions, diseases or disorders. In one aspect, the invention provides compounds of Formula IA and Formula IB, or its pharmaceutically acceptable salt or a prodrug or metabolite thereof

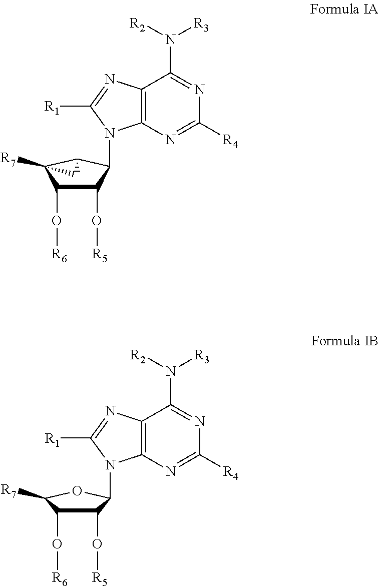

wherein
R₁ is selected from hydrogen, alkyl, alkenyl, alkynyl, aryl, heteroaryl wherein said alkyl, alkenyl, alkynyl, aryl, heteroaryl each are optionally substituted with one or more R₈;
R₂ is selected from hydrogen, amino acid, alkyl, alkenyl, alkynyl, heteroalkyl, aryl, arylalkyl, heteroaryl, heteroarylalkyl, cycloalkyl and heterocycloalkyl wherein said amino acid, alkyl, alkenyl, alkynyl, heteroalkyl, aryl, arylalkyl, heteroaryl, heteroarylalkyl, cycloalkyl and heterocycloalkyl each are optionally substituted with one or more R₈;
R₃ is selected from hydrogen, alkyl, aryl, arylalkyl, heteroaryl, heteroarylalkyl,

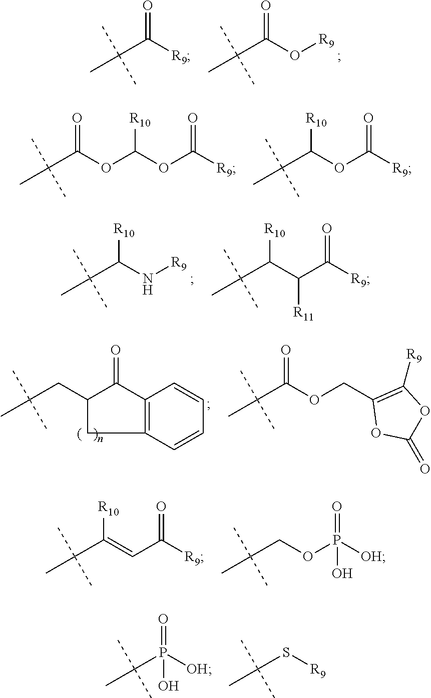

wherein n=1, 2, or 3;
R₄ is selected from hydrogen, amino acid, halogen, alkylthio, alkyl, alkenyl, alkynyl, heteroalkyl, aryl, arylalkyl, heteroaryl, heteroarylalkyl, cycloalkyl and heterocycloalkyl wherein said amino acid, alkyl, alkenyl, alkynyl, heteroalkyl, aryl, arylalkyl, heteroaryl, heteroarylalkyl, cycloalkyl and heterocycloalkyl each are optionally substituted with one or more R₈;
R₅ and R₆ are independently selected from hydrogen, alkyl,

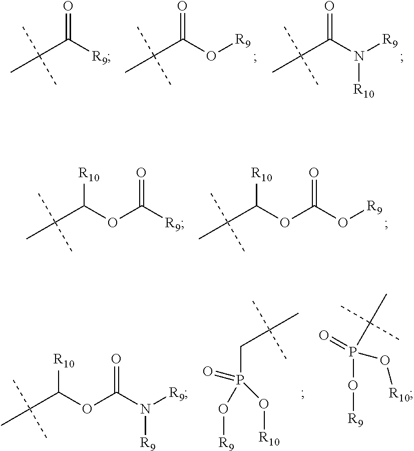

and R₅ and R₆ can be connected to form any of the following cyclic ring systems

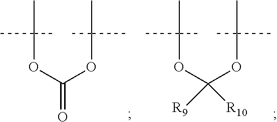

wherein R₉ and R₁₀ are selected from hydrogen, alkyl, heteroalkyl, aryl, arylalkyl, heteroaryl, heteroarylalkyl, cycloalkyl and heterocycloalkyl wherein said each are optionally substituted with one or more R₈;
and R₉ and R₁₀ can be connected to form a cycloalkyl or heterocycloalkyl include one or more nitrogen (N), oxygen (O) or sulfur (S) atoms; wherein said cycloalkyl, heterocycloalkyl are optionally substituted with one or more R₈;
R₇ is selected from hydrogen, alkyl,

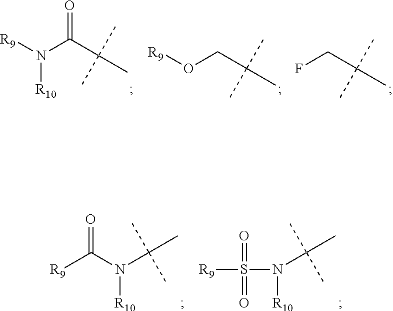

R₈ is independently selected for each occurrence from the group consisting of hydrogen, amino acid, lower alkyl, lower alkenyl, lower alkynyl, cycloalkyl, heterocycloalkyl aryl, heteroaryl, alicyclic, arylalkyl, heteroarylalkyl, lower alkoxy, aryloxy, amino, alkylamino, dialkylamino, diarylalkylamino, alkylthio, arylthio, heteroarylthio, oxo, oxa, —C(O)—R₁₂, —C(O)OR₁₂, —C(O)NR₁₂R₁₃, —C(S)NR₁₂R₁₃, CO₂H, acyloxy, halo, —CN, —NO₂, —N₃, —SH, —OH, —CH(CF₃)NR₁₂R₁₃, —C(═N)NR₁₂, perhaloalkyl, perhaloalkoxy, perhaloacyl, guanidinyl, pyridinyl, thiophene, furanyl, indolyl, indazolyl, phosphonate, phosphonic acid, phosphate, phosphoramide, sulfonate, sulfone, sulfate, sulphonamide, carbamate, urea, thiourea, thioamide and thioalkyl; wherein said heterocycloalkyl or heteroaryl include one or more nitrogen (N), oxygen (O) or sulfur (S) atoms; wherein R₁₂ and R₁₃ are independently selected from H, methyl, ethyl, and benzyl;
R₉, R₁₀, and R₁₁ are each independently selected from hydrogen, amino acid, lower alkyl, lower alkenyl, lower alkynyl, cycloalkyl, heterocycloalkyl aryl, heteroaryl, alicyclic, arylalkyl, heteroarylalkyl, lower alkoxy, aryloxy, —NO₂, —N₃, —SH, or —OH, each are optionally substituted with one or more R₈.

According to one embodiment, the invention also provides a compound of Formula IA and Formula IB, or its pharmaceutically acceptable salt or a prodrug or metabolite thereof wherein
R₁ is hydrogen;
R₃ is selected from

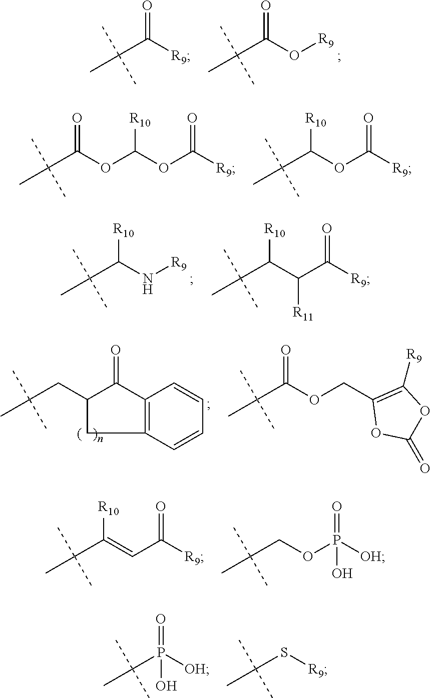

R₄ is halo;
R₇ is hydrogen.

According to one embodiment, the invention also provides a compound of Formula IA and Formula IB, or its pharmaceutically acceptable salt or a prodrug or metabolite thereof wherein
R₁ is Hydrogen;
R₃ is selected from

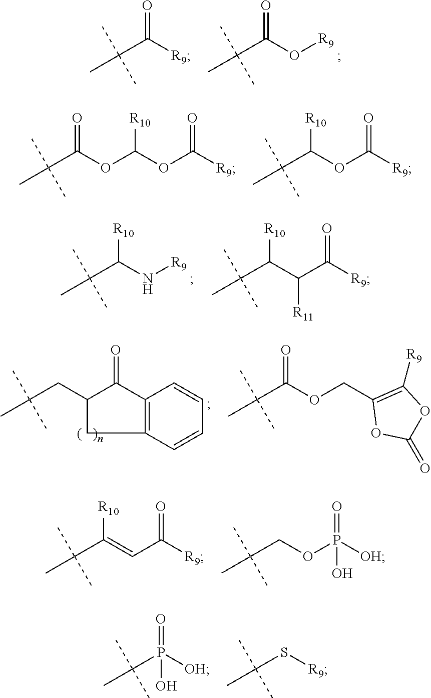

R₇ is

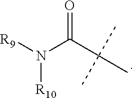

According to one embodiment, the invention also provides a compound of Formula II, or its pharmaceutically acceptable salt or a prodrug or metabolite thereof wherein

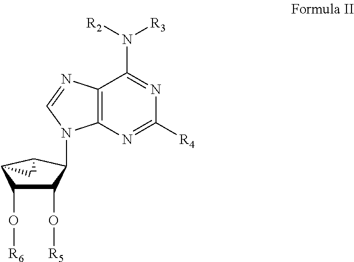

wherein R₂, R₃, R₄, R₅, and R₆ are as defined in claim 1.

According to one embodiment, the invention also provides a compound of Formula IIIA or Formula IIIB or its pharmaceutically acceptable salt or a prodrug or metabolite thereof

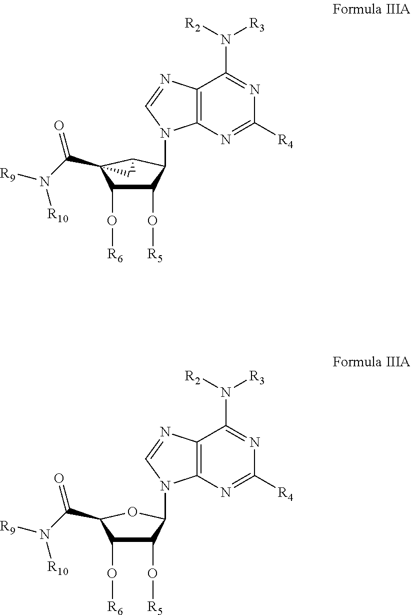

wherein R₂, R₃, R₄, R₅, R₆, R₉, and R₁₀ are as defined in claim 1.

According to one embodiment, the invention also provides a compound of Formula IV or its pharmaceutically acceptable salt or a prodrug or metabolite thereof

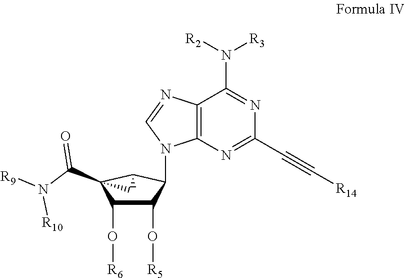

wherein R₁₄ is selected from aryl or heteroaryl which is optionally substituted with one or more R₈; and R₂, R₃, R₅, R₆, R₈, R₉, and R₁₀ are as defined in claim 1.

According to one embodiment, the invention also provides a compound of Formula VA or Formula VB or its pharmaceutically acceptable salt or a prodrug or metabolite thereof

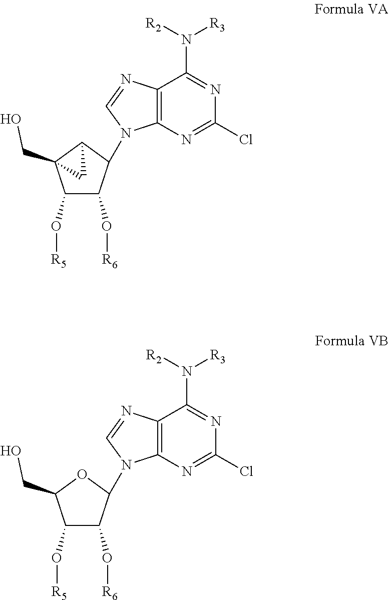

Wherein R₂, R₃, R₅, and R₆ are as defined in claim 1.

According to one embodiment, the invention also provides a compound of Formula VIA or Formula VIB or its pharmaceutically acceptable salt or a prodrug or metabolite thereof

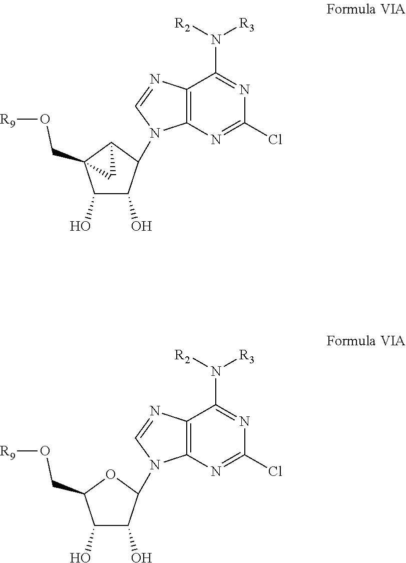

wherein R₂, R₃, and R₉ are as defined in claim 1.

According to one embodiment, the invention also provides a compound of Formula VII or its pharmaceutically acceptable salt or a prodrug or metabolite thereof

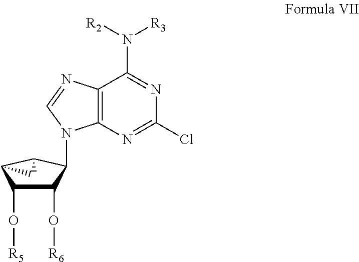

wherein R₂, R₃, R₅, and R₆ are as defined in claim 1.

According to one embodiment, the invention also provides a compound of Formula VIIIA or Formula VIIIB or its pharmaceutically acceptable salt or a prodrug or metabolite thereof

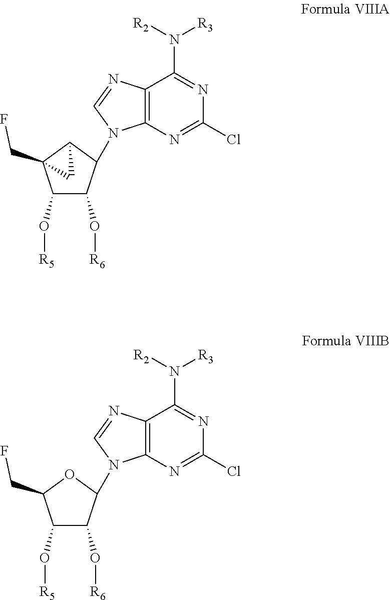

wherein R₂, R₃, R₅, and R₆ are as defined in claim 1.

According to one embodiment, the invention also provides a compound of Tables 1-14 or its pharmaceutically acceptable salt or a prodrug or metabolite thereof.

According to one embodiment, the invention also provides a pharmaceutical composition comprising a compound of Formula IA or Formula IB as defined in claim 1 or a pharmaceutically acceptable salt, ester or prodrug or metabolite thereof in association with a pharmaceutically acceptable diluent or carrier.

According to one embodiment, the invention also provides a pharmaceutical composition comprising a compound of Formula IA or Formula IB as defined in claim 1 or a pharmaceutically acceptable salt, ester or prodrug or metabolite thereof in association with a pharmaceutically acceptable diluent or carrier to form a formulation system for delivering the compound.

According to one embodiment, the invention also provides a pharmaceutical composition comprising a compound of Formula IA or Formula IB as defined in claim 1 or a pharmaceutically acceptable salt, ester or prodrug or metabolite thereof in a combination of other pharmaceutically active agent(s).

According to one embodiment, the invention also provides a compound of Formula IA or Formula IB as defined in claim 1 or a pharmaceutically acceptable salt, ester or prodrug or metabolite thereof for use in therapy.

According to one embodiment, the invention also provides use of a compound of Formula IA or Formula IB as defined in claim 1 or a pharmaceutically acceptable salt, ester or prodrug or metabolite thereof in the preparation of a medicament for the treatment of cardiovascular disease, nervous system disorders, Parkinson's disease, pain, neurodegenerative disorders, ischaemia and neuroprotection, sleep, renal system disorders, pulmonary disorders, inflammatory disorders, endocrine disorders, cancer, and visual disorders.

According to one embodiment, the invention also provides a process of producing a compound of Formula IA or Formula IB as defined in claim 1 or its pharmaceutically acceptable salt or prodrug or metabolite.

According to one embodiment, the invention also provides a process of producing Compound A as outlined in Scheme K and the procedures describing for producing Compound A.

According to one embodiment, the invention also provides a a modified-release formulation comprising compound of Formula IA and Formula IB a single active pharmaceutical ingredient, (a) at least one release controlling polymer selected from the group consisting of pH-dependent polymers and non-pH-dependent polymers, and (b) at least one stabilizer selected from the group consisting of acidifying agents and hydrophobizing agents, wherein the total amount of water content in the formulation is not more than 5% by weight of the formulation.

According to one embodiment, the invention also provides a modified-release formulation comprising compound A or Compound B a single active pharmaceutical ingredient, (a) at least one release controlling polymer selected from the group consisting of pH-dependent polymers and non-pH-dependent polymers, and (b) at least one stabilizer selected from the group consisting of acidifying agents and hydrophobizing agents.

According to one embodiment, the invention also provides a modified-release formulation comprising compound A a single active pharmaceutical ingredient, (a) at least one release controlling polymer selected from the group consisting of pH-dependent polymers and non-pH-dependent polymers, and (b) at least one stabilizer selected from the group consisting of acidifying agents and hydrophobizing agents, According to one embodiment, the invention also provides a modified-release formulation comprising compound B a single active pharmaceutical ingredient, (a) at least one release controlling polymer selected from the group consisting of pH-dependent polymers and non-pH-dependent polymers, and (b) at least one stabilizer selected from the group consisting of acidifying agents and hydrophobizing agents.

According to one embodiment, the invention also provides a modified-release formulation comprising compound A or compound B a single active pharmaceutical ingredient, in an enteric-coated capsules at least one stabilizer selected from the group consisting of acidifying agents and hydrophobizing agents.

According to one embodiment, the invention also provides a modified-release formulation comprising compound A a single active pharmaceutical ingredient, in an enteric-coated capsules at least one stabilizer selected from the group consisting of acidifying agents and hydrophobizing agents.

According to one embodiment, the invention also provides a modified-release formulation comprising compound B a single active pharmaceutical ingredient, in an enteric-coated capsules at least one stabilizer selected from the group consisting of acidifying agents and hydrophobizing agents.

According to one embodiment, a pharmaceutical composition comprising a compound of Formula IA or Formula IB or a pharmaceutically acceptable salt, ester or prodrug or metabolite thereof in association with a pharmaceutically acceptable diluent or carrier.

According to one embodiment, a pharmaceutical composition comprising a compound of Formula IA or Formula IB or a pharmaceutically acceptable salt, ester or prodrug or metabolite thereof in association with a pharmaceutically acceptable diluent or carrier to form a formulation system for delivering the compound.

According to one embodiment, a pharmaceutical composition comprising a compound of Formula IA or Formula IB as defined in claim 1 or a pharmaceutically acceptable salt, ester or prodrug or metabolite thereof in a combination of other pharmaceutically active agent(s).

According to one embodiment, a compound of Formula IA or Formula IB or a pharmaceutically acceptable salt, ester or prodrug or metabolite thereof for use in therapy.

According to one embodiment, the use of a compound of Formula IA or Formula IB or a pharmaceutically acceptable salt, ester or prodrug or metabolite thereof in the preparation of a medicament for the treatment of cardiovascular disease, nervous system disorders, Parkinson's disease, neurodegenerative disorders, ischaemia and neuroprotection, Sleep, renal system disorders, pulmonary disorders, inflammatory disorders, endocrine disorders, cancer, visual disorders.

According to one embodiment, a process of producing a compound of Formula IA or Formula IB or its pharmaceutically acceptable salt or prodrug or metabolite.

According to another aspect, a method for the treatment or prevention of a disease or condition modulated by GPCR(s) is provided and includes the step of administering a compound as provided herein. Any of the methods or uses provided herein may include administering to a subject a therapeutically effective amount of a compound as provided herein, including salt or polymorph thereof, or a pharmaceutical composition that includes such compounds.

The invention relates to formulation systems loaded with compounds of this invention mention above which said systems have improved biopharmaceutical properties for solubility, drug concentration in target tissue (s), in vivo efficacy and safety, improved quality (fineness and homogeneity of the particles, drug inclusion) and improved physical stability of the particulate formulation (no aggregation or gel formation). The compound of this invention can be appropriately formulated for desired delivery systems such as oral drug delivery (immediate release, delayed-release, prolonged release, modified release), parenteral drug delivery, ophthalmic drug delivery, nasal drug delivery, rectal drug delivery, colon-specific drug delivery, topical drug delivery, and CNS or brain drug delivery by powder injection; or by buccal, sublingual, or intranasal absorption. Pharmaceutical compositions may be formulated in unit dose form, or in multiple or subunit doses.

The manner in which the compounds or their pharmaceutical composition set forth herein may be administered can vary. According to one embodiment, the compounds can be administered orally. Preferred pharmaceutical compositions may be formulated for oral administration in the form of tablets, capsules, caplets, syrups, solutions, and suspensions. Such oral formulations can be provided in modified release dosage forms such as time-release tablet and capsule formulations. Pharmaceutical compositions can also be administered via injection, namely, intravenously, intramuscularly, subcutaneously, intraperitoneally, intra-arterial, intrathecally, and intracerebroventricularly.

Suitable carriers for injection are well known to those of skill in the art and include 5% dextrose solutions, saline, and phosphate-buffered saline.

According to another aspect, a formulation of the compound of the present invention can be prepared by entrapping the compound in liposomes, or albumin. The formulation of the compound of the present invention can be prepared by using nanoparticles, nanocapsules or nanospheres using appropriate excipient(s) such as cyclodextrins, mannitol, sodium dodecyl sulfate, albumin, polysorbate 80, trehalose, sucrose, lactose, tromethamine, sodium chloride, gelatin, amino acids.

Pharmaceutical compositions may also be administered using other means, for example, rectal administration. Formulations useful for rectal administration, such as suppositories, are well known to those of skill in the art. The compounds can also be administered by inhalation, for example, in the form of an aerosol; topically, such as, in lotion form; transdermally, such as, using a transdermal patch (for example, by using technology that is commercially available from Novartis and Alza Corporation); by powder injection; or by buccal, sublingual, or intranasal absorption. Pharmaceutical compositions may be formulated in unit dose form, or in multiple or subunit doses.

The administration of the pharmaceutical compositions described herein can be intermittent, or at a gradual, continuous, constant or controlled rate. The pharmaceutical compositions may be administered to a warm-blooded animal, for example, a mammal such as a human being. In addition, the time of day and the number of times per day that the pharmaceutical composition is administered can vary.

The compounds as provided herein may also be used for the preparation of a medicament for the treatment or prevention of a disease or condition modulated by GPCR(s). Methods for treating, preventing, delaying the onset of, or slowing the progression of disorders mediated by GPCR(s) involved in the regulation or dysregulation of gene expression, in mammals in need of such treatment are also provided. The methods involve administering to a subject a therapeutically effective amount of a compound as provided herein, including a salt thereof, or a pharmaceutical composition that includes such compounds.

According to one embodiment, the methods for treating, preventing, delaying the onset of, or slowing the progression of disorders mediated by acetylated proteins involved in the regulation or dysregulation of gene expression, in mammals in need of such treatment include the administration of at least one compound as provided herein including, but not limited to, the compounds provided according to Formula I.

The compounds alone or in a pharmaceutical composition as provided herein may be used in the treatment of a variety of disorders and conditions and, as such, may be used in combination with a variety of other suitable therapeutic agents useful in the treatment or prophylaxis of those disorders or conditions. Thus, one embodiment of the present disclosure includes the administration of the compound of the present disclosure in combination with other therapeutic compounds. Such a combination of pharmaceutically active agents may be administered together or separately and, when administered separately, the administration may occur simultaneously or sequentially, in any order. The amounts of the compounds or agents and the relative timings of administration will be selected in order to achieve the desired therapeutic effect. The administration in a combination of a compound of the present disclosure with other treatment agents may be in combination by administration concomitantly in (1) a unitary pharmaceutical composition including two or more compounds; or (2) separate pharmaceutical compositions each including one of the compounds. Alternatively, the combination may be administered separately in a sequential manner wherein one treatment agent is administered first and the other second. Such sequential administration may be close in time or remote in time.

Another aspect of the present disclosure includes combination therapy comprising administering to the subject a therapeutically or prophylactically effective amount of the compound of the present disclosure and one or more other therapy including chemotherapy, radiation therapy, gene therapy, or immunotherapy.

### Definitions

The following definitions are meant to clarify, but not limit, the terms defined. If a particular term used herein is not specifically defined, such term should not be considered indefinite. Rather, terms are used within their accepted meanings.

As used throughout this specification, the preferred number of atoms, such as carbon atoms, will be represented by, for example, the phrase “Cₓ-C_{y} alkyl,” which refers to an alkyl group, as herein defined, containing the specified number of carbon atoms. Similar terminology will apply for other preferred terms and ranges as well. Thus, for example, C₁₋₆ alkyl represents a straight or branched chain hydrocarbon containing one to six carbon atoms.

As used herein the term “alkyl” refers to a straight or branched chain hydrocarbon, which may be optionally substituted, with multiple degrees of substitution being allowed. The term “lower alkyl” refers to an alkyl that includes from one to six carbon atoms, Examples of “lower alkyl” as used herein include, but are not limited to, methyl, ethyl, propyl, isopropyl, isobutyl, n-butyl, tert-butyl, isopentyl, and n-pentyl.

As used herein, the term “alkene” or “alkenyl” group refers to an unsaturated hydrocarbon that includes one or more carbon-carbon double bonds. The term “lower alkene” refers to an alkene that includes from two to twenty carbon atoms, such as from two to ten carbon atoms. The term “substituted alkene” refers to an alkene that has one or more of its hydrogen atoms replaced by one or more substituent groups, such as halogen.

As used herein, the term “alkyne” or “alkynyl” group refers to an unsaturated hydrocarbon that includes one or more carbon-carbon triple bonds. The term “lower alkyne” refers to an alkyne that includes from two to twenty carbon atoms, such as from two to ten carbon atoms. The term “substituted alkyne” refers to an alkyne that has one or more of its hydrogen atoms replaced by one or more substituent groups, such as halogen.

As used herein, the term “cycloalkyl” refers to a fully saturated optionally substituted monocyclic, bicyclic, or bridged hydrocarbon ring, with multiple degrees of substitution being allowed. Preferably, the ring is three to twelve-membered, more preferably, from five- to six-membered. Exemplary “cycloalkyl” groups as used herein include, but are not limited to, cyclopropyl, cyclobutyl, cyclopentyl, cyclohexyl, and cycloheptyl.

As used herein, the term “alkoxy” refers to a group —OR^{a}, where R^{a} is “alkyl” as defined herein.

As used herein, the term “heterocycloalkyl” or “heterocycle” or “heterocyclyl” refers to an optionally substituted mono- or polycyclic ring system, optionally containing one or more degrees of unsaturation, and also containing one or more heteroatoms, which may be optionally substituted, with multiple degrees of substitution being allowed. Exemplary heteroatoms include nitrogen, oxygen, or sulfur atoms, including N-oxides, sulfur oxides, and carbon oxides.

Preferably, the ring is three to twelve-membered, preferably four, five or six-membered and is either fully saturated or has one or more degrees of unsaturation. Such rings may be optionally fused to one or more of another heterocyclic ring(s) or cycloalkyl ring(s). Examples of “heterocyclic” groups as used herein include, but are not limited to, tetrahydrofuran, pyran, tetrahydropyran, 1,4-dioxane, 1,3-dioxane, piperidine, pyrrolidine, morpholine, tetrahydrothiopyran, tetrahydrothiophene, pyrrolidinone, dihydrofuranone, thiazolidinone, azetidinone, cyclopentanone, piperidinone, thiomorpholinone, 2H-1,4-thiazin-3(4H)-one, dihydropyrimidine-2,4(1H,3H)-dione 1,4-dihydropyridine.

As used herein, the term “aryl” refers to a single benzene ring or fused benzene ring system which may be optionally substituted, with multiple degrees of substitution being allowed. Examples of “aryl” groups as used include, but are not limited to, phenyl, benzyl, 2-naphthyl, 1-naphthyl, anthracene, and phenanthrene. Preferable aryl rings have five- to ten-members. The term “aryl” also includes a fused benzene ring system, namely where a cyclic hydrocarbon or heterocycle (e.g., a cyclohexane or dioxane ring) or heteroaryl (e.g., pyridine) is fused with an aromatic ring (aryl, such as a benzene ring).

As used herein, the term “heteroaryl” refers to a monocyclic five to seven-membered aromatic ring, a fused bicyclic aromatic ring system comprising two of such aromatic rings, which may be optionally substituted, with multiple degrees of substitution being allowed, or to a fused bicyclic ring system namely where a cycloalkyl or heterocycle (e.g., a cyclohexane or dioxane ring) is fused with a heteroaryl ring. Preferably, heteroaryl rings contain five- to ten-members. These heteroaryl rings contain one or more nitrogen, sulfur, and/or oxygen atoms. In certain embodiments, the heteroaryl rings contain one to three nitrogen, one to three oxygen, or one or two sulfur atoms. N-oxides, sulfur oxides, and dioxides are permissible heteroatom substitutions. Examples of “heteroaryl” groups as used herein include, but are not limited to, furan, thiophene, pyrrole, imidazole, pyrazole, triazole, tetrazole, thiazole, oxazole, isoxazole, oxadiazole, thiadiazole, isothiazole, triazole, pyridine, pyridazine, pyrazine, pyrimidine, quinoline, isoquinoline, quinoxaline, benzofuran, benzoxazole, benzothiophene, indole, indazole, benzimidazole, imidazopyridine, pyrazolopyridine, and pyrazolopyrimidine.

As used herein the term “halogen” refers to fluorine, chlorine, bromine, or iodine.

As used herein the term “haloalkyl” refers to a substituted or unsubstituted alkyl group, as defined herein, that is substituted with at least one halogen. Examples of branched or straight chained “haloalkyl” groups as used herein include, but are not limited to, methyl, ethyl, propyl, isopropyl, n-butyl, and t-butyl substituted independently with one or more halogens, for example, fluoro, chloro, bromo, and iodo. The term “haloalkyl” should be interpreted to include such substituents as perfluoroalkyl groups such as —CF₃.

As used herein, the term “sulfhydryl” refers to refers to a —SH group.

As used herein, the term “alkylthio” refers to a group —SR^{a}, where R^{a} is “alkyl” as defined herein.

As used herein, the term “arylthio” refers to a group —SR^{a}, where R^{a} is “aryl” as defined herein.

As used herein, the term “carboxyamido” refers to —NH—C(O)—W, wherein W is hydrogen or an unsubstituted or substituted alkyl, alkene, alkyne, cycloalkyl, aryl, or heterocycle group.

As used herein, the term “amine” is given its ordinary meaning and includes primary, secondary, and tertiary amines.

As used herein, the term “amido” refers to a group of the formula —C(O)NR′R″, wherein R′ and R″ are substituted or unsubstituted alkyl, cycloalkyl or heterocycle, or R′ and R″ can form cycloalkyl or heterocycle. As used herein, the term “sulfamido” refers to the group —SO₂NR′R″.

As used herein, “optionally substituted”, groups may be substituted or unsubstituted. The substituent (or substitution) group may include, without limitation, one or more substituents independently selected from the following groups or designated subsets thereof: lower (C₁-C₆) alkyl, lower alkenyl, lower alkynyl, lower aryl, heteroaryl, alicyclic, heterocyclic, arylalkyl, heteroarylalkyl, lower alkoxy, lower aryloxy, amino, alkylamino, dialkylamino, diarylalkylamino, alkylthio, arylthio, heteroarylthio, oxo, oxa, carbonyl (—C(O)), carboxy esters (—C(O)OR), carboxamido (—C(O)NH₂), carboxy, acyloxy, —H, halo, —CN, —NO₂, —N₃, —SH, —OH, —C(O)CH₃, perhaloalkyl, perhaloalkoxy, perhaloacyl, guanidine, pyridinyl, thiophene, furanyl, indole, indazole, esters, amides, phosphonates, phosphonic acid, phosphates, phosphoramides, sulfonates, sulfones, sulfates, sulphonamides, carbamates, ureas, thioureas and thioamides, thioalkyls. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃).

As used herein, the term “pharmaceutically acceptable” refers to the carrier(s), diluent(s), excipient(s) or salt forms of the compounds of the present disclosure that are compatible with the other ingredients of the formulation of the pharmaceutical composition.

As used herein, the term “pharmaceutical composition” refers to a compound of the present disclosure optionally admixed with one or more pharmaceutically acceptable carriers, diluents, or excipients. Pharmaceutical compositions preferably exhibit a degree of stability to environmental conditions to make them suitable for manufacturing and commercialization purposes.

As used herein, the terms “effective amount”, “therapeutic amount”, and “effective dose” refer to an amount of the compound of the present disclosure sufficient to elicit the desired pharmacological or therapeutic effects, thus resulting in effective prevention or treatment of a disorder. Treatment of a disorder may be manifested by delaying or preventing the onset or progression of the disorder, as well as delaying or preventing the onset or progression of symptoms associated with the disorder. Treatment of a disorder may also be manifested by a decrease or elimination of symptoms, reversal of the progression of the disorder, as well as any other contribution to the well-being of the patient. The effective dose can vary, depending upon factors such as the condition of the patient, the severity of the symptoms of the disorder, and the manner in which the pharmaceutical composition is administered.

The term “prodrug” as used herein is intended to encompass a class of analogs of compounds of the present invention wherein a metabolically labile moiety is attached to said compound of the invention through an available NH, C(O)H, COOH, C(O)NH2, OH or SH functionality. The prodrug-forming moieties are removed by metabolic processes and release the active compounds having the free NH, C(O)H, COOH, C(O)NH2, OH or SH group in vivo.

Prodrugs are useful for adjusting such pharmacokinetic properties of the compounds as solubility and/or hydrophobicity, absorption in the gastrointestinal tract, bioavailability, tissue penetration, and rate of clearance. Design and preparation of such prodrugs are known to those skilled in the art, and are described in: Various forms of prodrugs are well known in the art and are described in:
a) The Practice of Medicinal Chemistry, Camille G. Wermuth et al., Ch. 31 (Academic Press, 1996).
b) Design of Prodrugs, edited by H. Bundgaard, (Elsevier, 1985); 33.
c) A Textbook of Drug Design and Development, P. Krogsgaard-Larson and H. Bundgaard, eds. Ch. 5, pp. 113-191 (Harwood Academic Publishers, 1991); and
d) Hydrolysis in Drug and Prodrug Metabolism, Bernard Testa and Joachim M. Mayer, (Wiley-VCH, 2003).
e) Prodrugs: challenges and rewards, Valentino J. Stella et al., Springer, 2007

Said references are incorporated herein by reference, particularly as to the description of prodrugs.

### General Methods for Preparation of Compounds

The present invention also provides a method for the synthesis of compounds of the present disclosure. The present invention further provides a method for the synthesis of compounds useful as intermediates in the preparation of compounds of the present disclosure. The compounds can be prepared according to the methods described below using readily available starting materials and reagents. In these reactions, variants may be employed which are themselves known to those of ordinary skill in this art but are not described in detail here. Those skilled in the art of organic synthesis will appreciate that there exist multiple means of producing compounds of the present disclosure. Illustrative synthetic methods, including those directed to specific, selected compounds noted in Tables 1-14, are as set forth herein.

It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by one of ordinary skill in the art by routine optimization procedures.

One skilled in the art of organic synthesis understands that vulnerable moieties such as C(O)OH, C(O) and C(O)H, NH, C(O)NH2, OH, and SH moieties may be protected and deprotected, as necessary. Protecting groups for C(O)OH moieties include, but are not limited to, allyl, benzoylmethyl, benzyl, benzyloxymethyl, tert-butyl, ethyl, methyl, 2,2,2-trichloroethyl, and the like. Protecting groups for C(O) and C(O)H moieties include, but are not limited to, 1,3-dioxylketal, diethylketal, dimethylketal, 1,3-dithianylketal, O-methyloxime, O-phenyloxime, and the like. Protecting groups for NH moieties include, but are not limited to, acetyl, benzoyl, benzyl(phenylmethyl), benzylidene, benzyloxycarbonyl (Cbz), tert-butoxycarbonyl (Boc), 3,4-dimethoxybenzyloxycarbonyl, diphenylmethyl, diphenylphosphoryl, formyl, methanesulfonyl, para-methoxybenzyloxycarbonyl, phenylacetyl, phthaloyl, succinyl, trichloroethoxycarbonyl, triethylsilyl, trifluoroacetyl, trimethylsilyl, triphenylmethyl, triphenylsilyl, para-toluenesulfonyl and the like.

Protecting groups for NH, OH and SH moieties include, but are not limited to, acetyl, allyl, allyloxycarbonyl, benzyloxycarbonyl (Cbz), benzoyl, benzyl, tert-butyl, tert-butyldimethylsilyl, tert-butyldiphenylsilyl, 3,4-dimethoxybenzyl, 3,4-dimethoxybenzyloxycarbonyl, 1,1-dimethyl-2-propenyl, diphenylmethyl, methanesulfonyl, methoxyacetyl, 4-methoxybenzyloxycarbonyl, para-methoxybenzyl, methoxycarbonyl, methyl, para-toluenesulfonyl, 2,2,2-trichloroethoxycarbonyl, 2,2,2-trichloroethyl, triethylsilyl, trifluoroacetyl, 2-(trimethylsilyl)ethoxycarbonyl, 2-trimethylsilylethyl, triphenylmethyl, 2-(triphenylphosphonio)ethoxycarbonyl and the like.

A discussion of protecting groups is provided in T. H. Greene and P. G. M. Wuts, Protective Groups in Organic Synthesis, 3rd Ed., John Wiley & Sons, New York (1999).

The invention will now be further described with reference to the following illustrative examples in which, unless stated otherwise: (i) temperatures are given in degrees Celsius (° C.); operations are carried out at room temperature (RT) or ambient temperature, that is, in a range of 18-25° C.; (ii) organic solutions were dried over anhydrous sodium or magnesium sulfate unless otherwise stated; evaporation of organic solvent was carried out using a rotary evaporator under reduced pressure; (iii) column chromatography means flash chromatography on silica gel; thin layer chromatography (TLC) was carried out on silica gel plates; (iv) in general, the course of reactions was followed by TLC or liquid chromatography/mass spectroscopy (LC/MS) and reaction times are given for illustration only; (v) final products have satisfactory proton nuclear magnetic resonance (NMR) spectra and/or mass spectra data; (vi) yields are given for illustration only and are not necessarily those which can be obtained by diligent process development; preparations were repeated if more material was required; (vii) when given, NMR data is in the form of delta values for major diagnostic protons, given in part per million (ppm) relative to tetramethylsilane (TMS) as an internal standard, and were obtained in the solvent indicated; (viii) chemical symbols have their usual meanings; (ix) in the event that the nomenclature assigned to a given compound does not correspond to the compound structure depicted herein, the structure will control; (x) solvent ratio is given in volume:volume (v/v) terms. The chemical names of the compounds and drawing of the chemical formulas are produced using ChemOffice professional. The abbreviation for chemicals, solvents, reagents, and protecting groups are used as a common practice in the chemistry field.

Compounds of claim 1 of the current invention can be prepared according to Schemes A-P as set forth below.

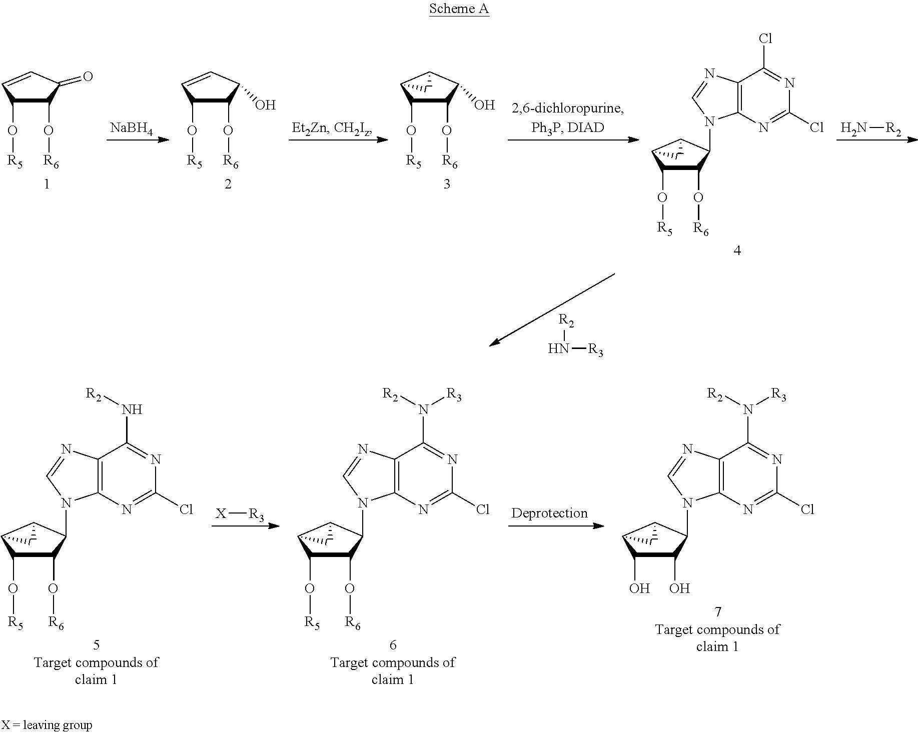

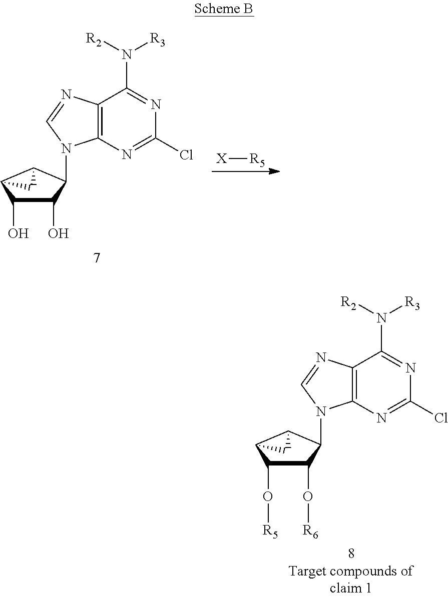

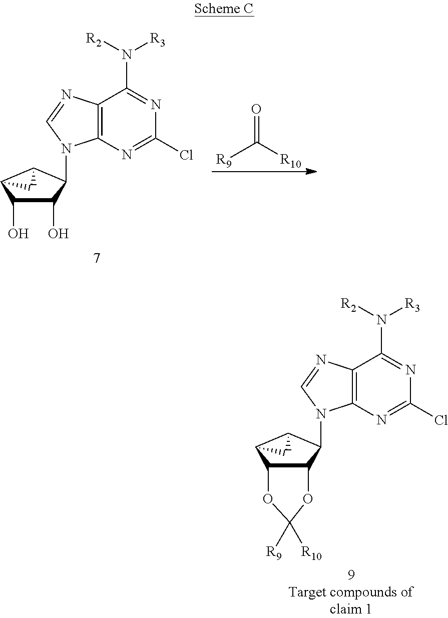

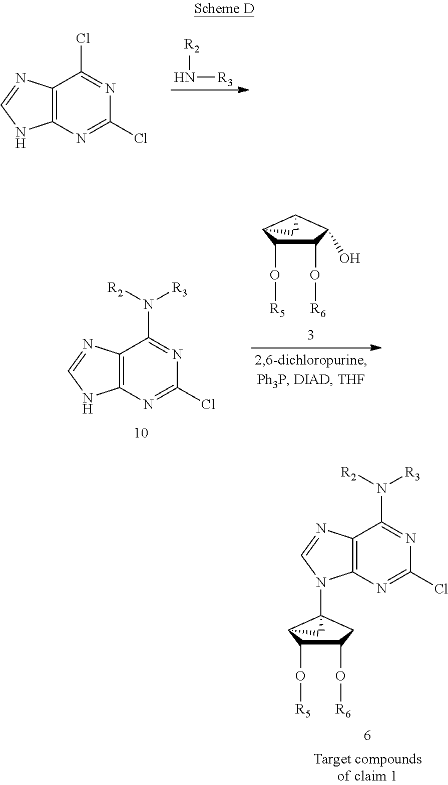

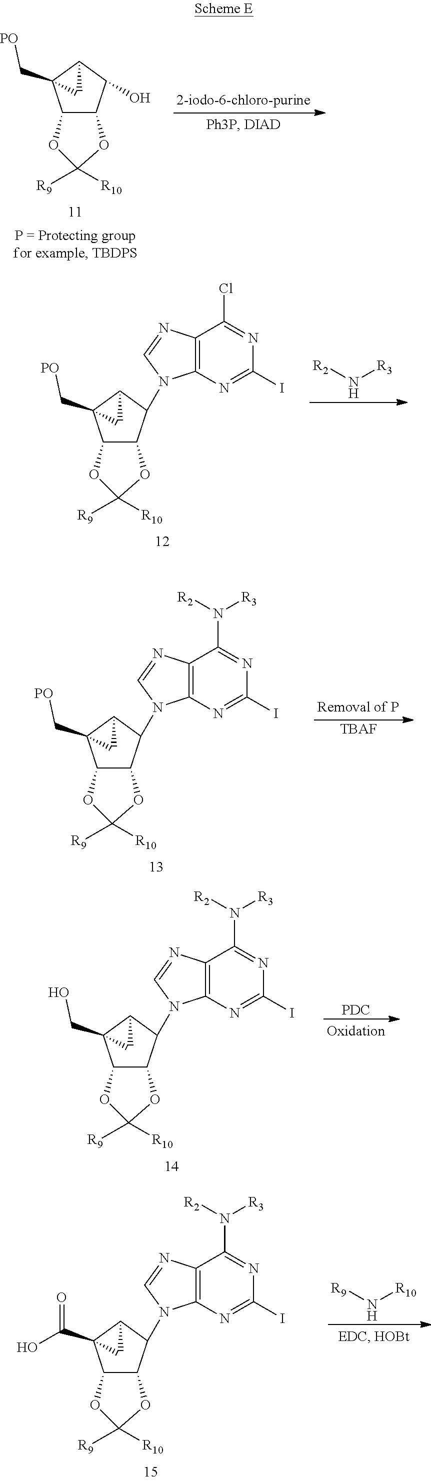

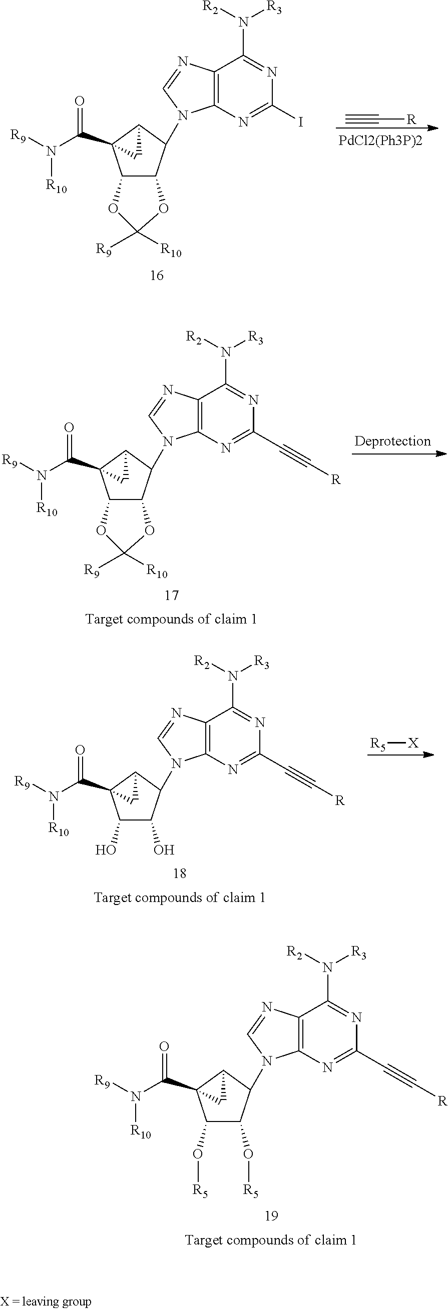

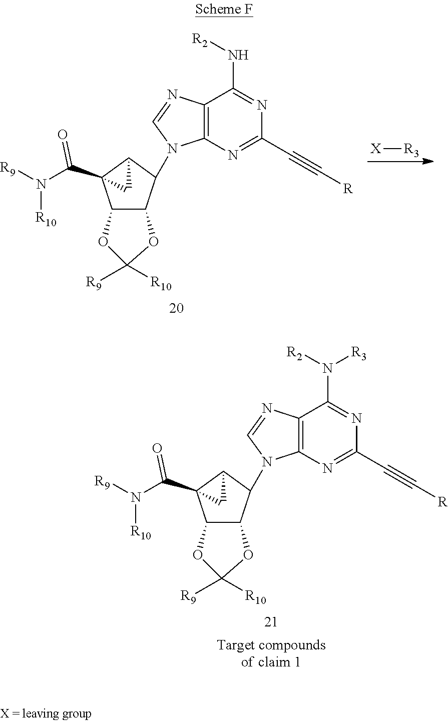

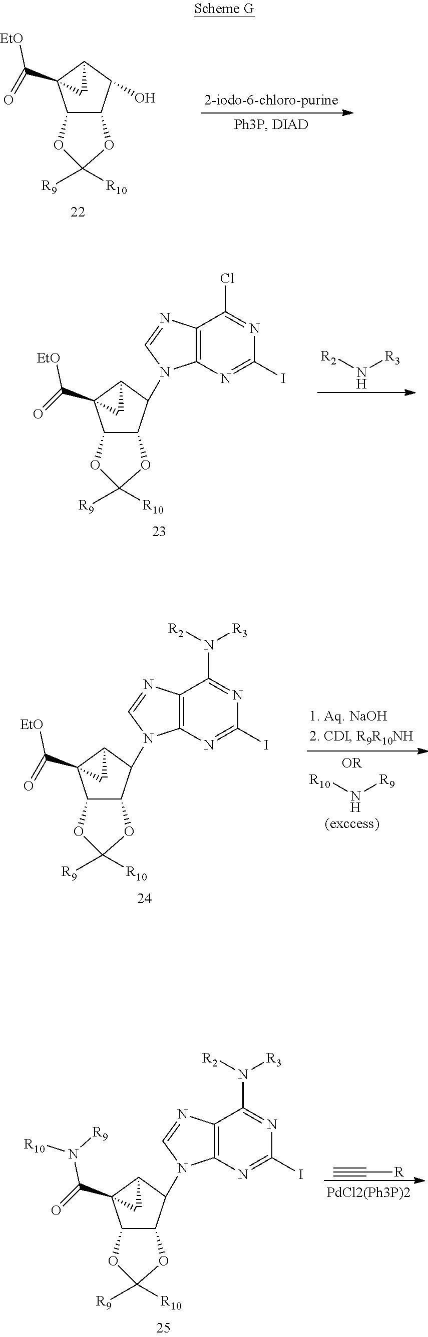

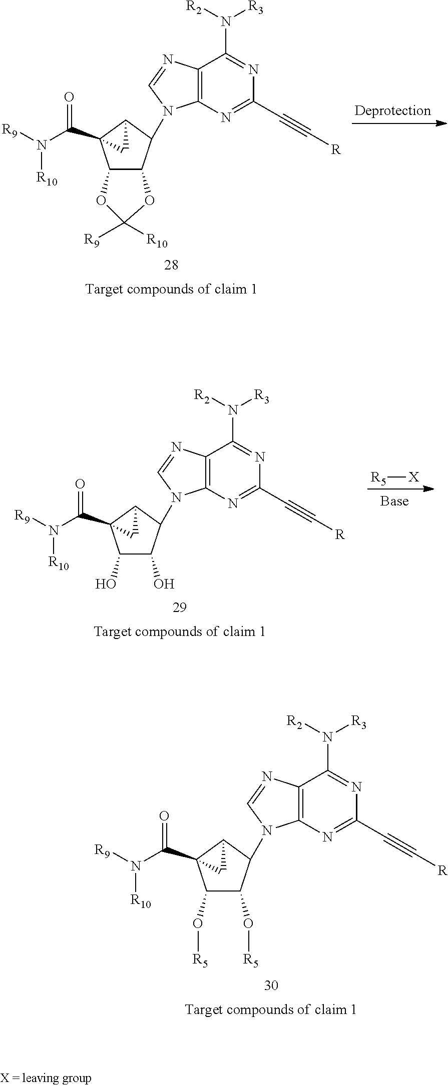

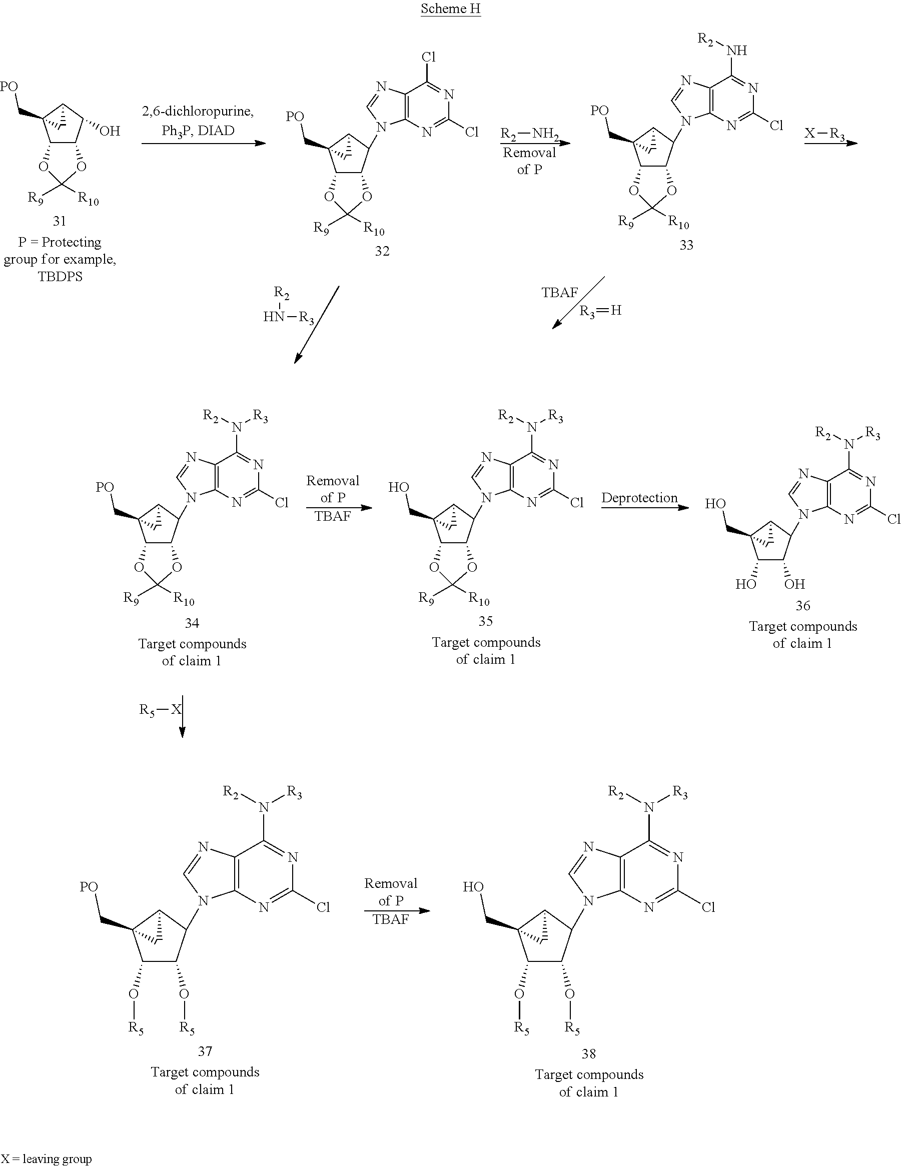

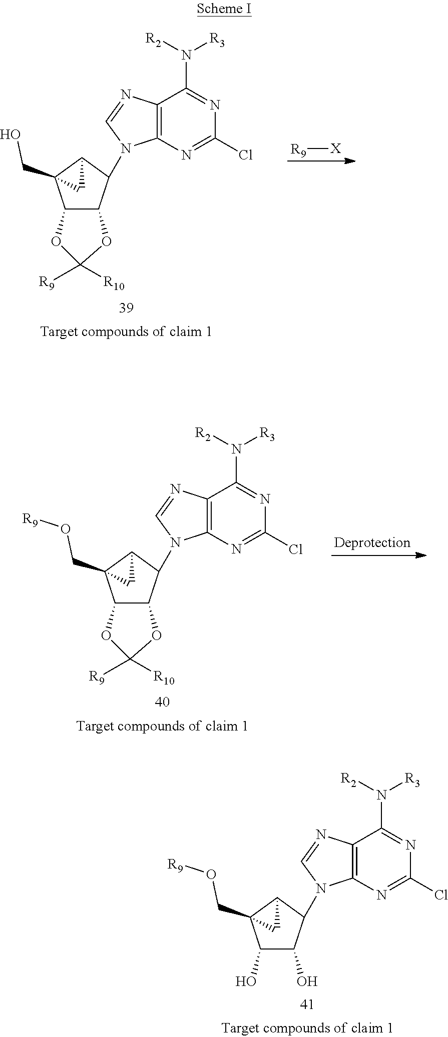

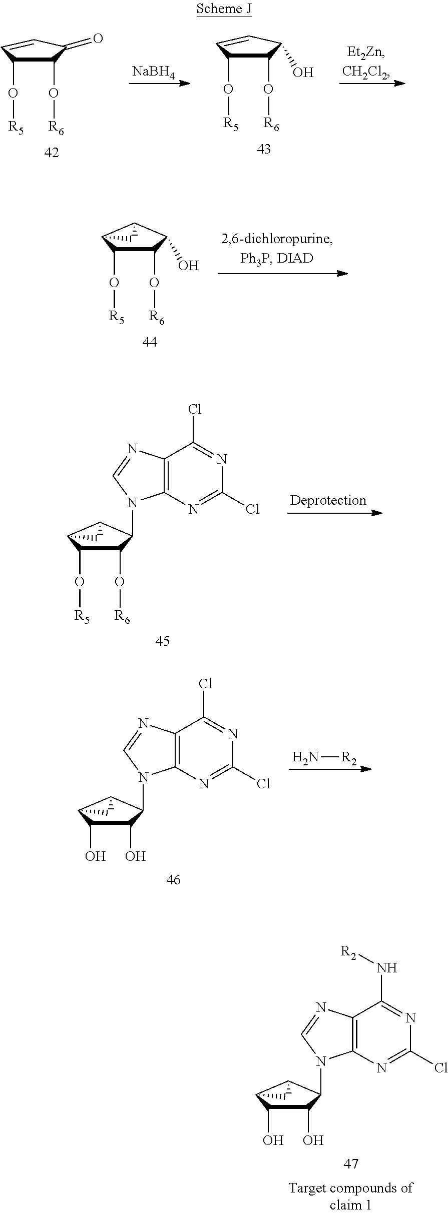

R₁, R₂, R₃, R₄, R₅, R₆, R₇, R₈, R₉, and R₁₀ in the above schemes are as defined above. R is selected from alkyl, cycloalkyl, heterocycloalkyl, aryl and heteroaryl, wherein said heteroaryl or heterocycloalkyl include one or more nitrogen (N), oxygen (O) or sulfur (S) atoms; wherein said alkyl, cycloalkyl, heterocycloalkyl, aryl and heteroaryl are optionally substituted with one or more R₁₀.

It is appreciated and understand that functionality on any of the R, R₁, R₂, R₃, R₄, R₅, R₆, R₇, R₈, R₉, and R₁₀ may be required to be protected to carry out reaction(s) and then finally deprotected to obtain the final product. The final compounds may be obtained in the racemic mixture, and then each constituent isomers can be purified by either crystallization or chiral chromatography.

### EXAMPLES

### Compound A: (1R,2R,3S,4R,5S)-4-(2-chloro-6-((dicyclopropylmethyl)amino)-9H-purin-9-yl)bicyclo[3.1.0]hexane-2,3-diol

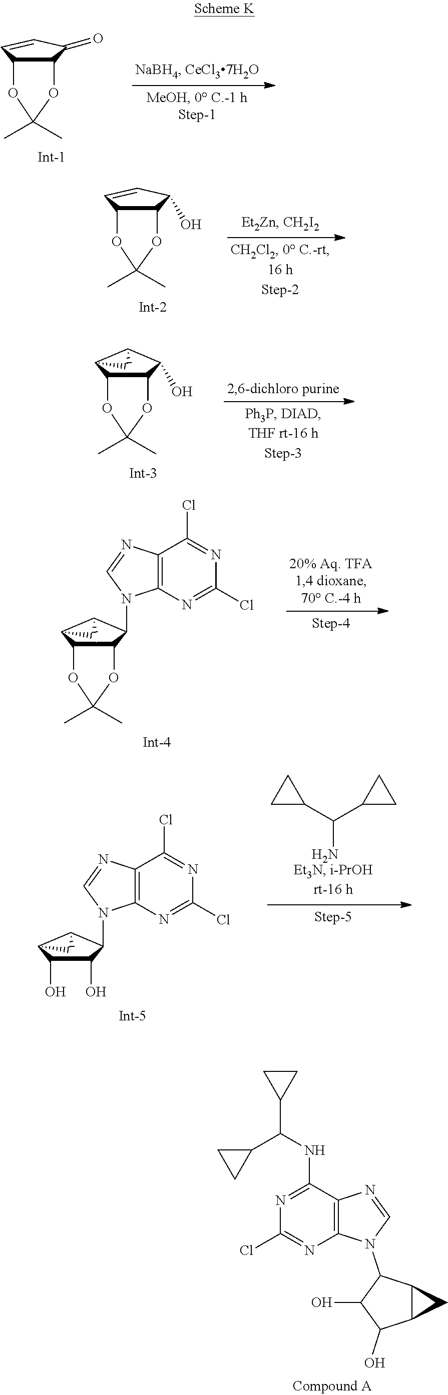

### Step-1: (3aS,4S,6aR)-2,2-dimethyl-3a,6a-dihydro-4H-cyclopenta[d][1,3]dioxol-4-ol

To a stirred solution of (3aR,6aR)-2,2-dimethyl-3a,6a-dihydro-4H-cyclopenta[d][1,3]dioxol-4-one (Int-1) (200 g, 1.29 mol) in methanol (4 L) at 0° C. was portion wise added NaBH₄ (98.65 g, 2.596 mol) and stirred at room temperature for 1 h. Methanol was evaporated under vacuum and the resulting residue was stirred in ethyl acetate (2 L) for 1 h, filtered through celite pad and the filtrate was concentrated under vacuum. The process was repeated for one more time and obtained (160 g, 79%) of (3aS,4S,6aR)-2,2-dimethyl-3a,6a-dihydro-4H-cyclopenta[d][1,3]dioxol-4-ol (Int-2) as a pale yellow liquid. HNMR (FIG. 1).

### Step-2: (3aS,4S,6aR)-2,2-dimethyl-3a,6a-dihydro-4H-cyclopenta[d][1,3]dioxol-4-ol)

To a solution of (3aS,4S,6aR)-2,2-dimethyl-4,6a-dihydro-3aH-cyclopenta[d][1,3]dioxol-4-ol (2) (150 g, 0.961 mol) in CH₂Cl₂ (3 L) at 0° C. was added drop wise Et₂Zn (1.92 L, 1.92 mol, 1 M in hexane) and stirred at same temp for 15 min. To this reaction mixture was added, drop wise, CH₂I₂ (310 mL, 3.84 mmol) and stirred at room temperature for 16 h. The reaction mixture was quenched with saturated aq. ammonium chloride solution and extracted with CH₂Cl₂ (2×3 L). The combined CH₂Cl₂ layer was washed with brine (2 L), dried over Na₂SO₄ and concentrated under vacuum. The crude compound was purified by column chromatography {silica gel (100-200); EtOAc/pet-ether 25%} to get (3aR,3bR,4aS,5S,5aS)-2,2-dimethylhexahydrocyclopropa[3,4]cyclopenta[1,2-d][1,3]dioxol-5-ol (Int-3) (96 g, 59%) as a pale brown liquid. HNMR (FIG. 2)

### Step-3: 2,6-dichloro-9-((3aR,3bR,4aS,5R,5aS)-2,2-dimethylhexahydro cyclopropa[3,4]cyclopenta[1,2-d][1,3]dioxol-5-yl)-9H-purine (Int-4)

To a solution of 2,6-dichloro-9H-purine (133.4 g, 0.70 mol) and PPh₃ (185 g, 0.70 mol) in THF (1.6 L) at 0° C. was added drop wise DIAD (148 mL, 0.75 mol) and stirred at same temperature for 10 min. To this reaction mixture was added (3aR,3bR,4aS,5S,5aS)-2,2-dimethylhexahydrocyclopropa[3,4]cyclopenta[1,2-d][1,3]dioxol-5-ol (Int-3) (80 g, 0.470 mol) and stirred at rt for 16 h. The reaction mixture was evaporated under vacuum and the crude residue was purified by column chromatography [Silica gel (100-200)]; EtOAc: Pet-ether 30%) to get 2,6-dichloro-9-((3aR,3bR,4aS,5R,5aS)-2,2-dimethylhexahydrocyclopropa[3,4]cyclopenta[1,2-d][1,3]dioxol-5-yl)-9H-purine (Int-4) (70 g, 44%) as an off-white sticky solid. HNMR (FIG. 3)

### Step-4: (1R,2R,3S,4R,5S)-4-(2,6-dichloro-9H-purin-9-yl)bicyclo[3.1.0]hexane-2,3-diol (Int-5)

To a solution of 2,6-dichloro-9-((3aR,3bR,4aS,5R,5aS)-2,2-dimethylhexahydrocyclopropa[3,4]cyclopenta[1,2-d][1,3]dioxol-5-yl)-9H-purine (Int-4) (80 g, 234.6 mmol) in 1,4 dioxane (240 mL) was added 20% aq. TFA (240 mL) and the resulting mixture was then heated to 70° C. and stirred there for 4 h. The reaction mixture was evaporated under vacuum and neutralised with saturated aq. NaHCO₃ solution and extracted with EtOAc (2×500 mL). The combined EtOAc layer was washed with brine (200 mL), dried over Na₂SO₄, concentrated under vacuum. The crude product was purified by column chromatography [Silica gel (100-200); MeOH-DCM; 4%) to get (1R,2R,3S,4R,5S)-4-(2,6-dichloro-9H-purin-9-yl)bicyclo[3.1.0]hexane-2,3-dioI (Int-5) (45 g, 64%) as an off white solid. HNMR and LCMS FIG. 4

### Step-5: (1R,2R,3S,4R,5S)-4-(2-chloro-6-((dicyclopropylmethyl)amino)-9H-purin-9-yl)bicyclo[3.1.0]hexane-2,3-diol (Compound A)

To a solution of (1R,2R,3S,4R,5S)-4-(2,6-dichloro-9H-purin-9-yl)bicyclo[3.1.0]hexane-2,3-diol (Int-5) (80 g, 265.78 mmol) and Et₃N (55.2 mL, 398.67 mmol) in i-PrOH (800 mL) was added dicyclopropylmethanamine (32.21 g, 318.93 mmol) and stirred at room temperature for 16 h. The reaction mixture was concentrated under vacuum and residue was diluted with water (500 mL), extracted with CH₂Cl₂ (2×500 mL). The combined CH₂Cl₂ layer was washed with brine (300 mL), dried over Na₂SO₄ and concentrated under vacuum. The crude compound was purified by column chromatography [silica gel (100-200); MeOH/CH₂Cl₂ 3%), further purified by GRACE flash chromatography (silica gel; 3% MeOH/CH₂Cl₂), re-precipitated with CH₂Cl₂/N-pentane and finally co-distilled with ethanol to get of (1R,2R,3S,4R,5S)-4-(2-chloro-6-((dicyclopropylmethyl)amino)-9H-purin-9-yl)bicycle[3.1.0]hexane-2,3-diol (Compound A) (54 g, 54%) as an off-white solid. HNMR and LCMS FIG. 5.

### Compound T1.1: (1R,5S)-4-(2-chloro-6-((dicyclopropylmethyl)amino)-9H-purin-9-yl)bicyclo[3.1.0]hexane-2,3-diyl (2S,2'S)-bis(2-amino-3-methylbutanoate), dihydrochloride salt

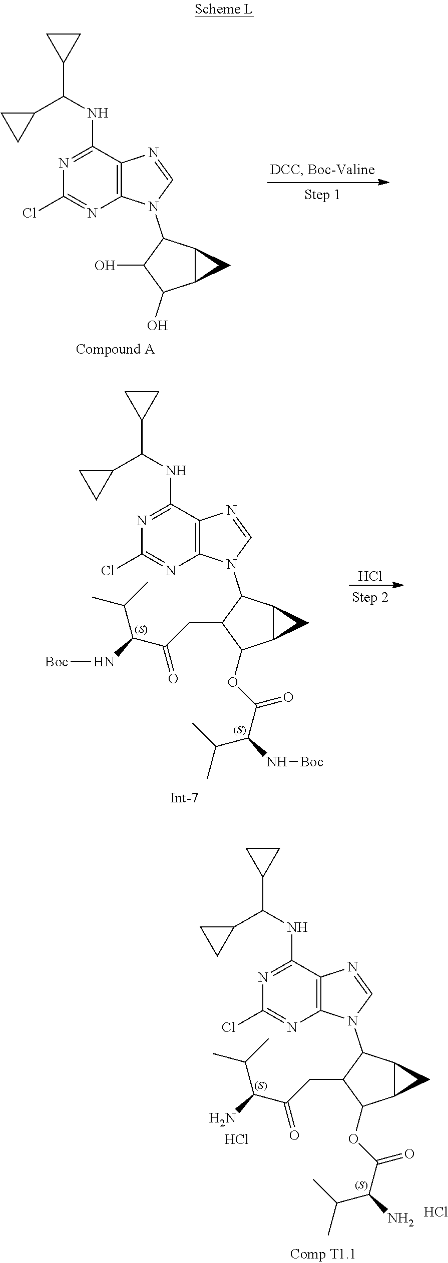

### Int-7:

Step 1: To a stirred reaction mixture of Boc-Valine (425 mg) and trimethylamine (100 microliters) and HOBt (300 mg) in DCM (10 mL) was added DCC (450 mg) at room temperature for 30 minutes and Compound A (375 mg) was added. The reaction mixture was stirred at room temperature for 16 hours and then concentrated. After aqueous work up, the organic layer was concentrated. The solid residue was purified by silica gel column chromatography using ethyl acetate-hexanes to yield light yellow solid (150 mg) of Int-7. Mass spec (M+H) 673.3

### Comp T1.1:

The product obtained in step 1 (Int-7 100 mg) was dissolved in ethanol, and 100 microliters of 1 N HCl was added. The reaction mixture was stirred for 16 hours at room temperature and then concentrated to provide a yellow solid of the target compound T1.1. Mass spec (M+H) 574.3

### Compound T2.8: 2-chloro-N-(dicyclopropylmethyl)-9-((3bR,4aS)-2-(1-methylpiperidin-4-yl)hexahydrocyclopropa[3,4]cyclopenta[1,2-d][1,3]dioxol-5-yl)-9H-purin-6-amine

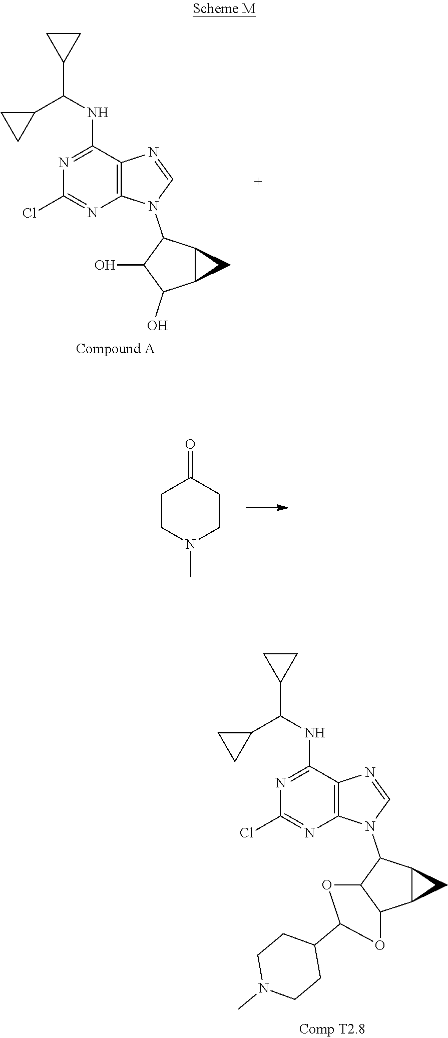

A reaction mixture of Compound A (375 mg), 1-methylpiperidin-4-one (120 mg) and p-toluenesufonic acid (25 mg) in toluene (25 mL) was refluxed at 125 C using dean and Stark instrument for 24 hours. The reaction mixture was concentrated and purified using silica gel chromatography to yield 105 mg of the desired product Compound T2.8. (M+H 485.3)

### Compound T3.1: (S)-2-amino-N-(2-chloro-9-((1S,2R,3S,4R,5R)-3,4-dihydroxybicyclo[3.1.0]hexan-2-yl)-9H-purin-6-yl)-N-(dicyclopropylmethyl)-3-methylbutanamide

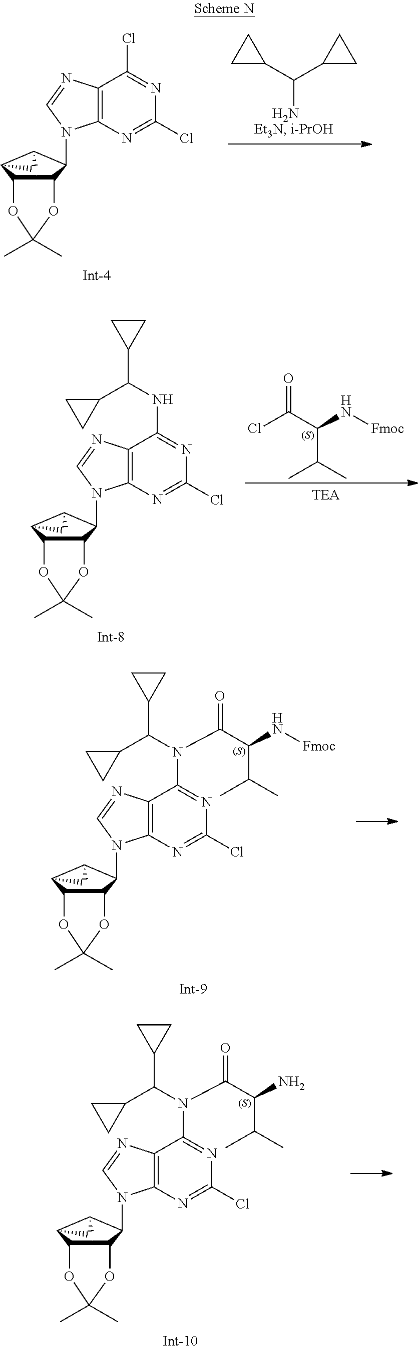

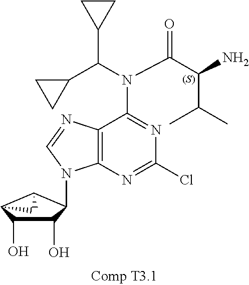

### Int-8:

A reaction mixture of Int-4 (680 mg), dicyclopropylmethanamine (250 mg) and triethylamine (300 mg) in isopropyl alcohol (25 mL) for 36 hours. The reaction mixture was concentrated and was purified by using silica gel chromatography to get Int-8 (510 mg). M+H 416.2

### Int-9

A reaction mixture of Int-8 (415 mg), (9H-fluoren-9-yl)methyl (S)-(1-chloro-3-methyl-1-oxobutan-2-yl)carbamate (400 mg) and triethylamine (200 mg) in DCM (20 mL) for 10 hours. The reaction mixture was concentrated and was purified by using silica gel chromatography to get Int-9 (456 mg). M+H 737.3

### Int-10

A reaction mixture of Int-9 (400 mg), piperidine (100 mg) in DCM (10 mL) for 4 hours. The reaction mixture was concentrated and was purified by using silica gel chromatography to get Int-8 (115 mg). M+H 515.3

### Comp T3.1

A reaction mixture of Int-10 (90 mg), in 1,4 dioxane (5 mL) was added 20% aq. TFA 2.0 mL) and the resulting mixture was then heated to 60° C. and stirred there for 4 h. The reaction mixture was evaporated under vacuum and neutralized with saturated aq. NaHCO₃ solution and extracted with ethyl acetate and concentrated. The crude product was purified by column chromatography to get the desired compound T3.1 (55 mg). M+H 475.2

### Compound T4.1: ((1R,2R,3S,4R,5S)-4-(2-chloro-6-((dicyclopropylmethyl)amino)-9H-purin-9-yl)-2,3-dihydroxybicyclo[3.1.0]hexan-1-yl)methyl L-valinate

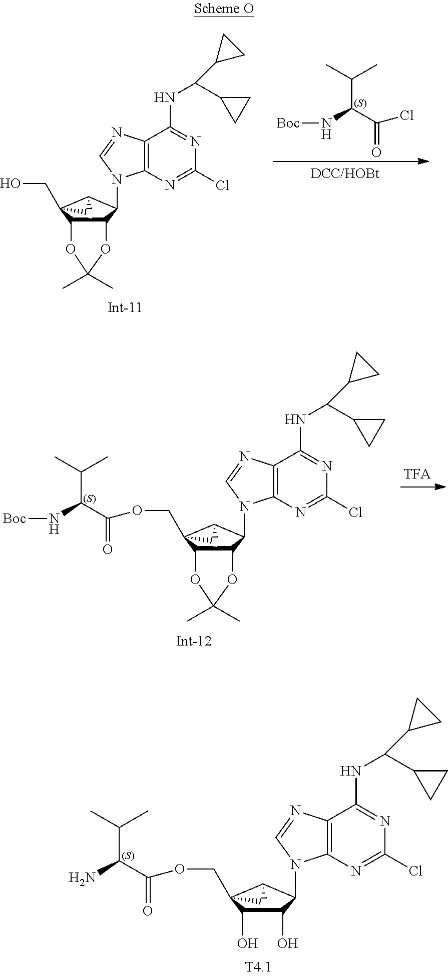

Int-12: ((3aR,3bR,4aS,5R,5aS)-5-(2-chloro-6-((dicyclopropylmethyl)amino)-9H-purin-9-yl)-2,2-dimethyltetrahydrocyclopropa[3,4]cyclopenta[1,2-d][1,3]dioxol-3b(3aH)-yl)methyl (tert-butoxycarbonyl)-L-valinate

A reaction mixture of Int-8 (445 mg), tert-butyl (S)-(1-chloro-3-methyl-1-oxobutan-2-yl)carbamate (300 mg) and triethylamine (200 mg) in DCM (20 mL) for 10 hours. The reaction mixture was concentrated and was purified by using silica gel chromatography to get Int-12 (359 mg). M+H 645.3

### T4.1

A reaction mixture of Int-12 (190 mg), in 1,4 dioxane (5 mL) was added 20% aq. TFA 1.2 mL) and the resulting mixture was then heated to 60° C. and stirred there for 6 h. The reaction mixture was evaporated under vacuum and neutralized with saturated aq. NaHCO₃ solution and extracted with ethyl acetate and concentrated. The crude product was purified by column chromatography to get the desired compound T3.1 (55 mg). M+H 505.2

### Compound T8.1: (1S,2R,3S,4R,5S)-4-(6-((3-chlorobenzyl)amino)-2-((3,4-difluorophenyl)ethynyl)-9H-purin-9-yl)-1-(methylcarbamoyl)bicyclo[3.1.0]hexane-2,3-diyl (2S,2'S)-bis(2-amino-3-methylbutanoate)

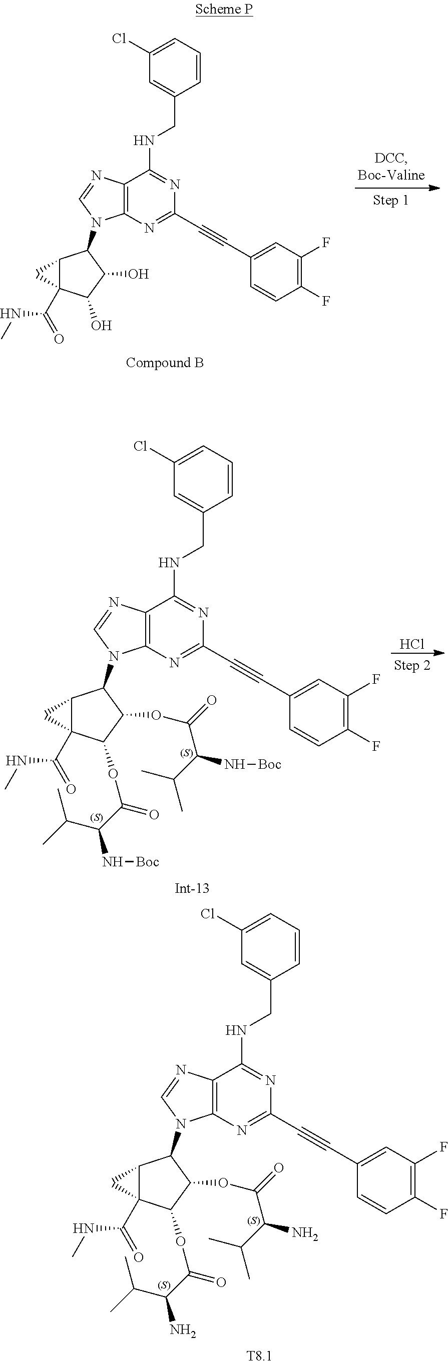

### Int-13

Step 1: To a stirred reaction mixture of Boc-Valine (450 mg) and trimethylamine (100 microliters) and HOBt (270 mg) in DCM (10 mL) was added DCC (450 mg) at room temperature for 30 minutes, and Compound B (564 mg) was added. The reaction mixture was stirred at room temperature for 18 hours and then concentrated. After aqueous work up, the organic layer was concentrated. The solid residue was purified by silica gel column chromatography using ethyl acetate-hexanes to yield white solid (250 mg) of Int-7. Mass spec (M+H) 963.4

### Comp T8.1:

Step 2: The product obtained in step 1 (Int-13, 200 mg) was dissolved in ethanol, and 100 microliters of 1 N HCl was added. The reaction mixture was stirred for 16 hours at room temperature and then concentrated to provide a dark yellow solid of the target compound T1.1.

Mass spec (M+H) 763.3.

### Compound T9.8: (3aR,3bS,4aS,5R,5aS)-5-(6-((3-chlorobenzyl)amino)-2-((3,4-difluorophenyl)ethynyl)-9H-purin-9-yl)-N,1′-dimethyltetrahydrospiro[cyclopropa[3,4]cyclopenta[1,2-d][1,3]dioxole-2,4′-piperidine]-3b(3aH)-carboxamide

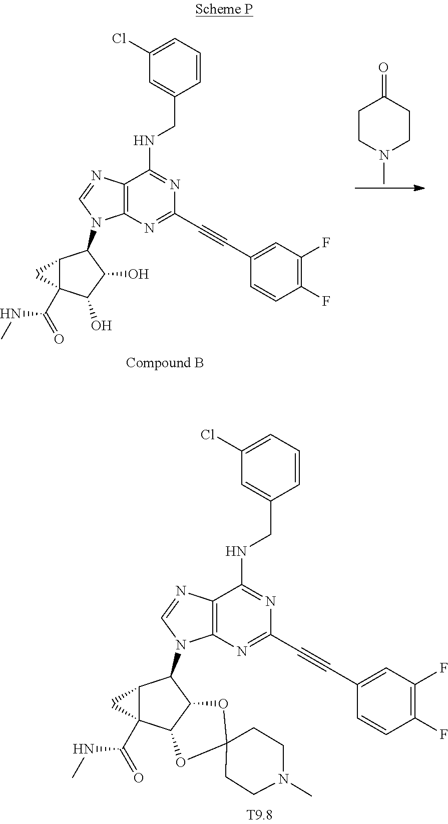

A reaction mixture of Compound 6 (400 mg), 1-methylpiperidin-4-one (100 mg) and p-toluenesufonic acid (20 mg) in toluene (25 mL) was refluxed at 125 C using Dean and Stark instrument for 36 hours. The reaction mixture was concentrated and purified using silica gel chromatography to yield 175 mg of the desired product Compound T9.8. (M+H 660.3)

Following compounds in Tables 1-14 can be made using a procedure similar to those outlined the schemes A-P.

TABLE 1

| | |
|---|---|
| | Formula A |
| 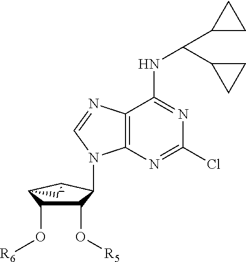 | |

| No. | R₅ | R₆ |
|---|---|---|
| T1.1 | 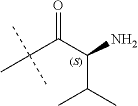 | 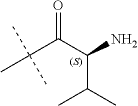 |
| T1.2 | 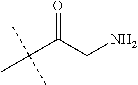 | 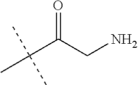 |
| T1.3 | 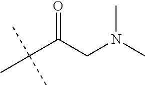 | 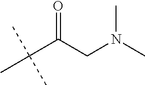 |
| T1.4 | 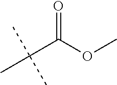 | 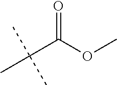 |
| T1.5 | 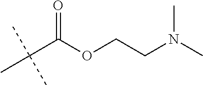 | 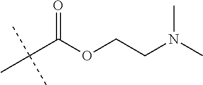 |
| T1.6 | 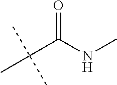 | 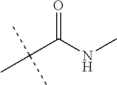 |
| T1.7 | 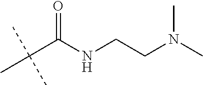 | 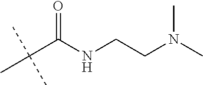 |
| T1.8 | 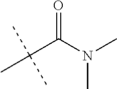 | 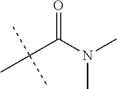 |
| T1.9 | 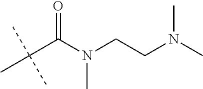 | 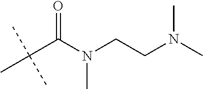 |
| T1.10 | 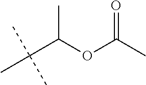 | 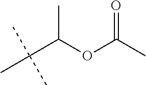 |
| T1.11 | 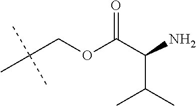 | 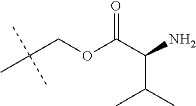 |
| T1.12 | 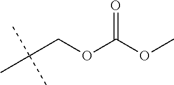 | 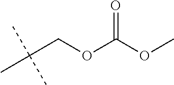 |
| T1.13 | 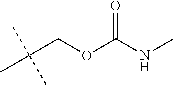 | 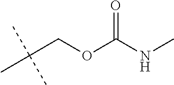 |
| T1.14 | 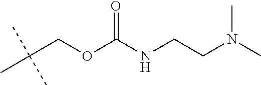 | 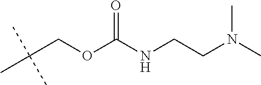 |
| T1.15 | 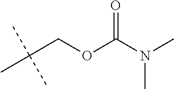 | 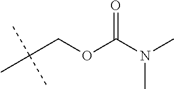 |
| T1.16 | 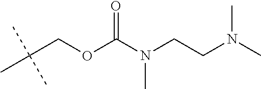 | 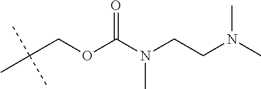 |
| T1.17 | 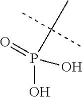 | 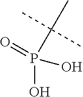 |
| T1.18 | 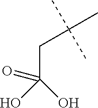 | 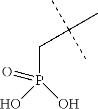 |
| T1.19 | 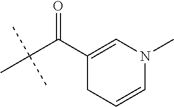 | 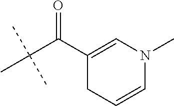 |
| T1.20 | H | Me |
| T1.21 | Me | H |

TABLE 2

| | |
|---|---|
| | Formula B |
| 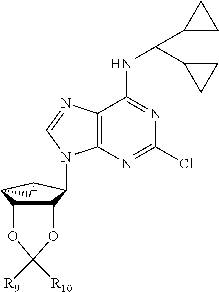 | |

| No. | R₉ | R₁₀ |
|---|---|---|
| T2.1 | H | H |
| T2.2 | —CH₃ | —CH₃ |
| T2.3 | H | —CH₃ |
| T2.4 | H | —Ph |
| T2.5 | H | 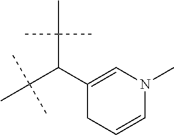 |

| | |
|---|---|
| | R₉R₁₀ |
| T2.6 | 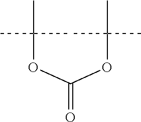 |
| T2.7 | 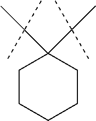 |
| T2.8 | 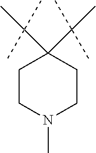 |

TABLE 3

| | |
|---|---|
| | Formula C |
| 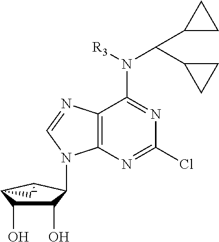 | |

| No. | R3 |
|---|---|
| T3.1 | 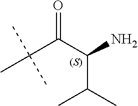 |
| T3.2 | 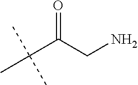 |
| T3.3 | 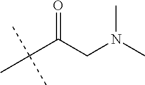 |
| T3.4 | 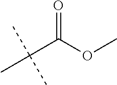 |
| T3.5 | 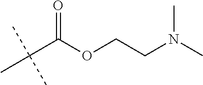 |
| T3.6 | 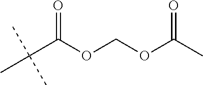 |
| T3.7 | 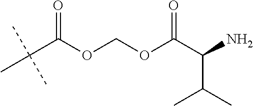 |
| T3.8 | 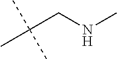 |
| T3.9 | 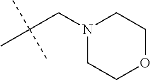 |
| T3.10 | 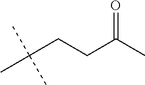 |
| T3.11 | 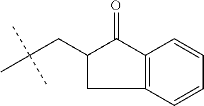 |
| T3.12 | 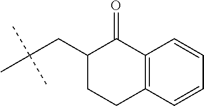 |
| T3.13 | 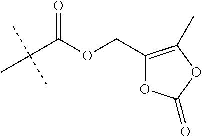 |
| T3.14 | 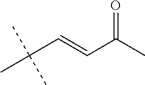 |
| T3.15 | 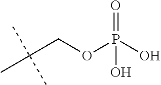 |
| T3.16 | 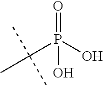 |
| T3.17 | 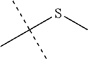 |

TABLE 4

| | |
|---|---|
| | Formula D |
| 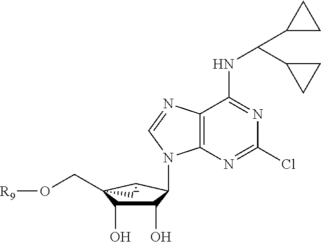 | |

| No. | R₉ |
|---|---|
| T4.1 | 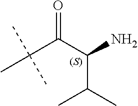 |
| T4.2 | 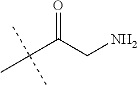 |
| T4.3 | 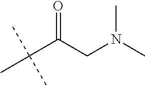 |
| T4.4 | 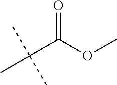 |
| T4.5 | 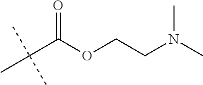 |
| T4.6 | 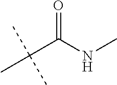 |
| T4.7 | 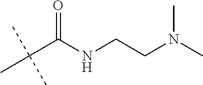 |
| T4.8 | 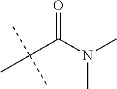 |
| T4.9 | 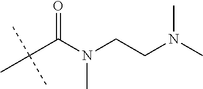 |
| T4.10 | 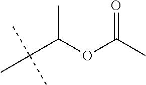 |
| T4.11 | 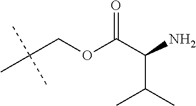 |
| T4.12 | 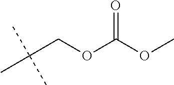 |
| T4.13 | 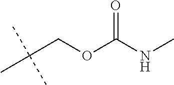 |
| T4.14 | 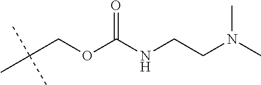 |
| T4.15 | 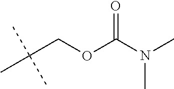 |
| T4.16 | 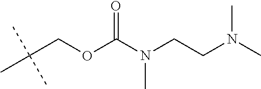 |
| T4.17 | 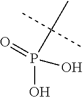 |
| T4.18 | 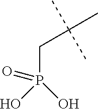 |
| T4.19 | 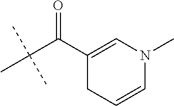 |

TABLE 5

| | |
|---|---|
| | Formula E |
| 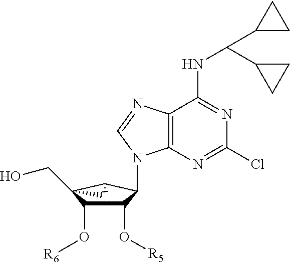 | |

| No. | R₅ | R₆ |
|---|---|---|
| T5.1 | 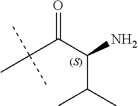 | 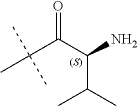 |
| T5.2 | 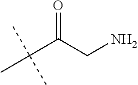 | 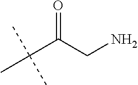 |
| T5.3 | 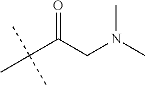 | 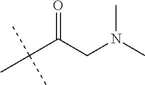 |
| T5.4 | 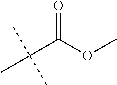 | 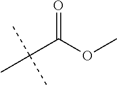 |
| T5.5 | 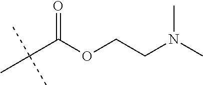 | 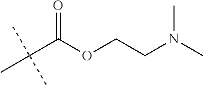 |
| T5.6 | 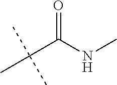 | 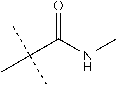 |
| T5.7 | 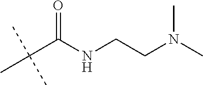 | 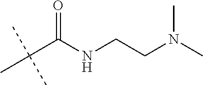 |
| T5.8 | 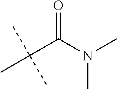 | 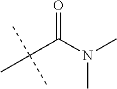 |
| T5.9 | 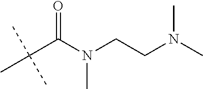 | 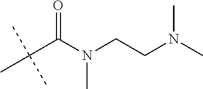 |
| T5.10 | 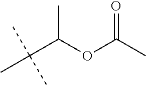 | 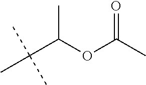 |
| T5.11 | 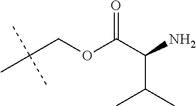 | 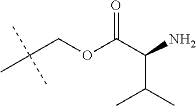 |
| T5.12 | 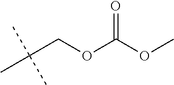 | 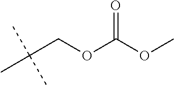 |
| T5.13 | 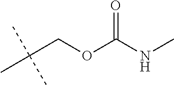 | 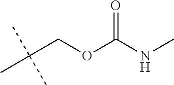 |
| T5.14 | 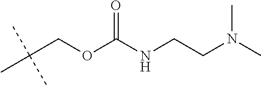 | 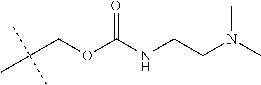 |
| T5.15 | 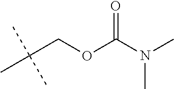 | 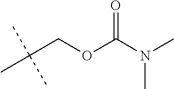 |
| T5.16 | 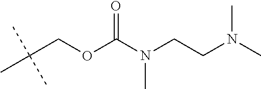 | 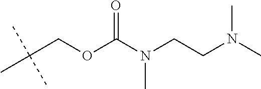 |
| T5.17 | 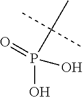 | 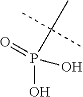 |
| T5.18 | 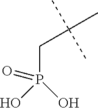 | 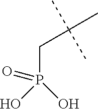 |
| T5.19 | 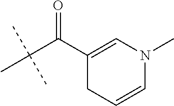 | 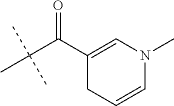 |
| T5.20 | H | Me |
| T5.21 | Me | H |

TABLE 6

| | |
|---|---|
| | Formula G |
| 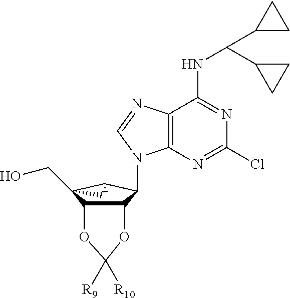 | |

| No. | R₉ | R₁₀ |
|---|---|---|
| T5.1 | H | —H |
| T5.2 | —CH3 | —CH3 |
| T5.3 | H | —CH3 |
| T5.4 | H | —Ph |
| T5.5 | H | 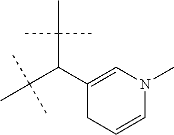 |

| | |
|---|---|
| | R₉R₁₀ |
| T5.6 | 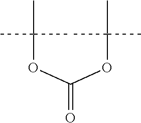 |
| T5.7 | 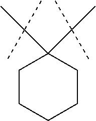 |
| T5.8 | 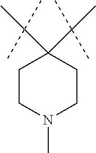 |

TABLE 7

| | |
|---|---|
| | Formula H |
| 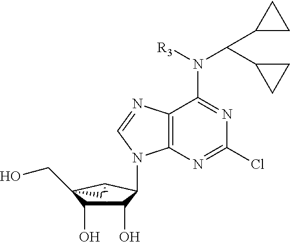 | |

| No. | R₃ |
|---|---|
| T7.1 | 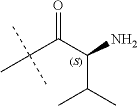 |
| T7.2 | 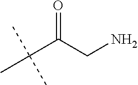 |
| T7.3 | 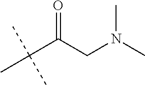 |
| T7.4 | 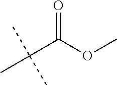 |
| T7.5 | 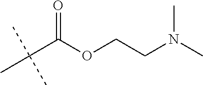 |
| T7.6 | 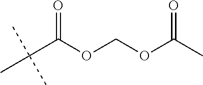 |
| T7.7 | 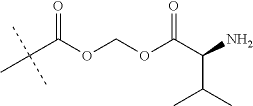 |
| T7.8 | 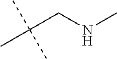 |
| T7.9 | 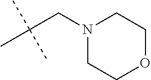 |
| T7.10 | 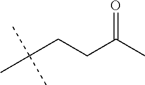 |
| T7.11 | 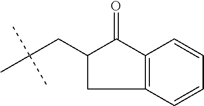 |
| T7.12 | 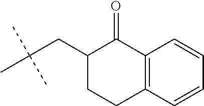 |
| T7.13 | 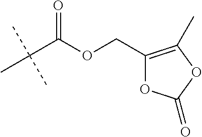 |
| T7.14 | 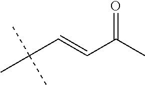 |
| T7.15 | 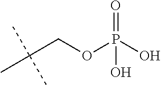 |
| T7.16 | 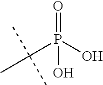 |
| T7.17 | 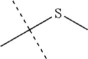 |

TABLE 8

| | |
|---|---|
| | Formula I |
| 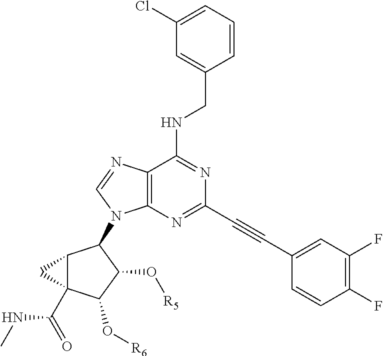 | |

| No. | R₅ | R₆ |
|---|---|---|
| T8.1 | 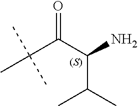 | 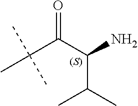 |
| T8.2 | 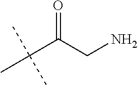 | 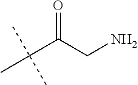 |
| T8.3 | 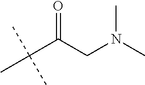 | 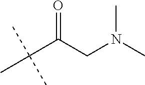 |
| T8.4 | 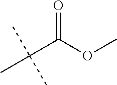 | 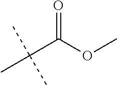 |
| T8.5 | 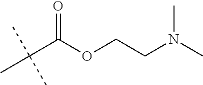 | 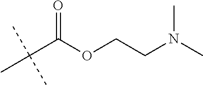 |
| T8.6 | 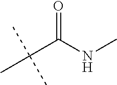 | 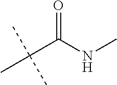 |
| T8.7 | 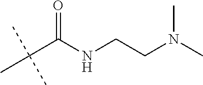 | 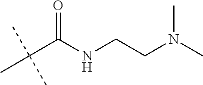 |
| T8.8 | 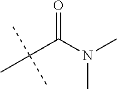 | 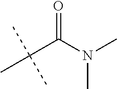 |
| T8.9 | 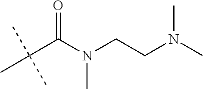 | 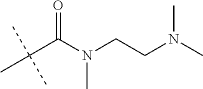 |
| T8.10 | 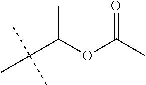 | 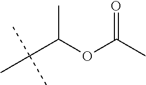 |
| T8.11 | 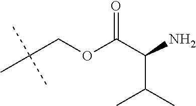 | 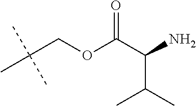 |
| T8.12 | 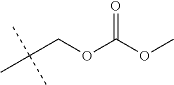 | 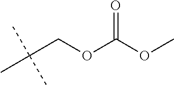 |
| T8.13 | 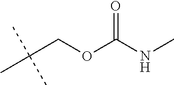 | 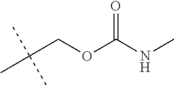 |
| T8.14 | 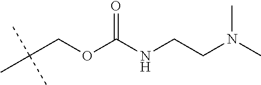 | 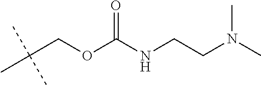 |
| T8.15 | 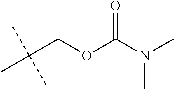 | 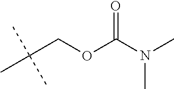 |
| T8.16 | 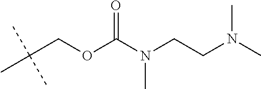 | 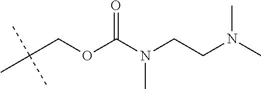 |
| T8.17 | 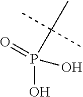 | 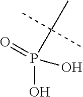 |
| T8.18 | 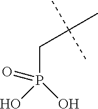 | 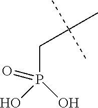 |
| T8.19 | 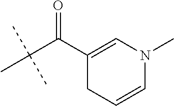 | 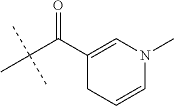 |
| T8.20 | H | Me |
| T8.21 | Me | H |

TABLE 9

| | |
|---|---|
| | Formula J |
| 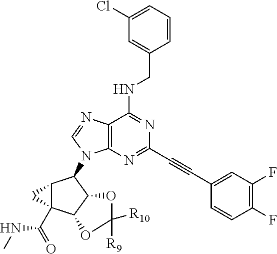 | |

| No. | R₉ | R₁₀ |
|---|---|---|
| T9.1 | H | H |
| T9.2 | —CH₃ | —CH₃ |
| T9.3 | H | —CH₃ |
| T9.4 | H | —Ph |
| T9.5 | H | 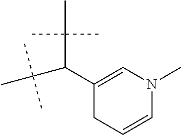 |

| | |
|---|---|
| | R₉R₁₀ |
| T9.6 | 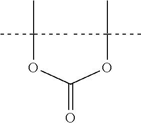 |
| T9.7 | 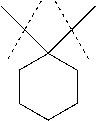 |
| T9.8 | 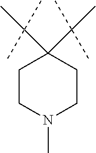 |

TABLE 10

| | |
|---|---|
| | Formula K |
| 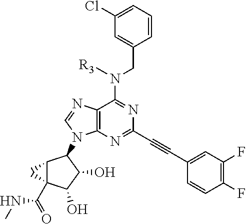 | |

| No. | R₃ |
|---|---|
| T10.1 | 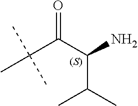 |
| T10.2 | 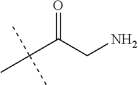 |
| T10.3 | 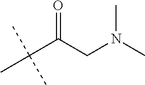 |
| T10.4 | 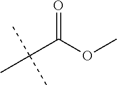 |
| T10.5 | 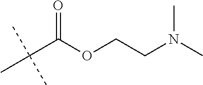 |
| T10.6 | 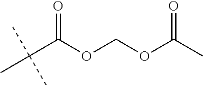 |
| T10.7 | 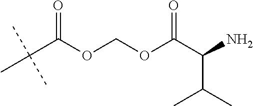 |
| T10.8 | 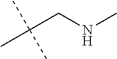 |
| T10.9 | 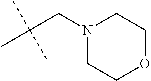 |
| T10.10 | 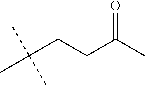 |
| T10.11 | 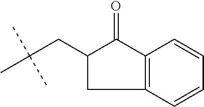 |
| T10.12 | 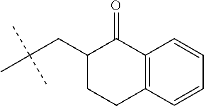 |
| T10.13 | 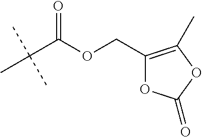 |
| T10.14 | 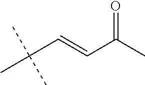 |
| T10.15 | 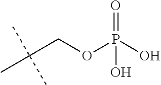 |
| T10.16 | 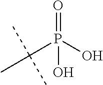 |
| T10.17 | 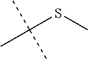 |

TABLE 11

| | |
|---|---|
| | Formula L |
| 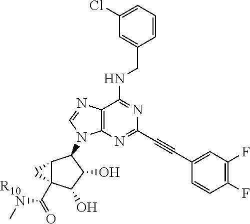 | |

| No. | R₁₀ |
|---|---|
| T11.1 | 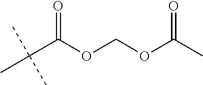 |
| T11.2 | 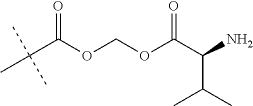 |
| T11.3 | 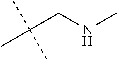 |
| T11.4 | 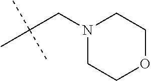 |
| T11.5 | 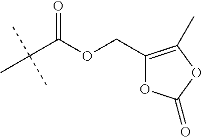 |
| T11.6 | 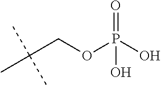 |
| T11.7 | 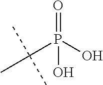 |
| T11.8 | 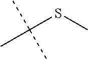 |

TABLE 12

| | |
|---|---|
| 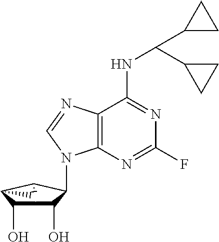 | Compound T12.1 |
| 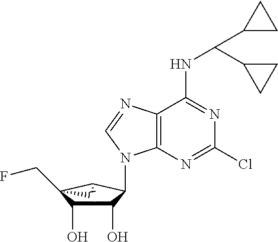 | Compound T12.1 |
| 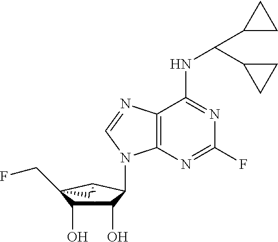 | Compound T12.1 |

TABLE 13

| | |
|---|---|
| | Formula M |
| 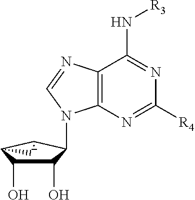 | |

| No. | R₃ | R₄ |
|---|---|---|
| T13.1 | 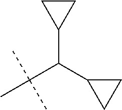 | Cl |
| T13.2 | 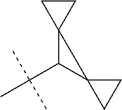 | Cl |
| T13.3 | 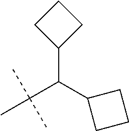 | Cl |
| T13.4 | 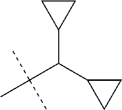 | H |
| T13.5 | 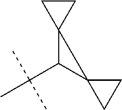 | H |
| T13.6 | 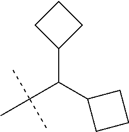 | H |

TABLE 14

| | |
|---|---|
| | Formula N |
| 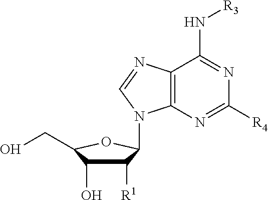 | |

| No. | R₃ | R₄ |
|---|---|---|
| T14.1 | 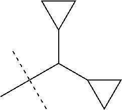 | Cl |
| T14.2 | 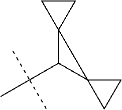 | Cl |
| T14.3 | 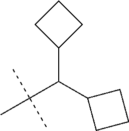 | Cl |
| T14.4 | 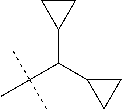 | H |
| T14.5 | 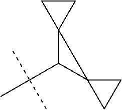 | H |
| T14.6 | 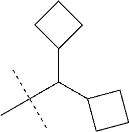 | H |

TABLE 15
Binding affinities for Adenosine receptors
The assay was performed as described in Chinese hamster ovary (CHO)
cells stably expressing either the recombinant hA1 or hA3AR and
human embryonic kidney (HEK) 293 cells stably expressing the
human (h) A2AAR were cultured in DMEM and F12 (1:1), supplemented
with 10% fetal bovine serum, 100 units/mL penicillin, 100 μg/mL
streptomycin, and 2 μmol/mL glutamine. In addition, 800 μg/mL
Geneticin and 500 μg/mL hygromycin were added to the A2A media
and to the A1 and A3 media, respectively. After harvesting the
cells, they were homogenized for 10 sec with an electric homogenizer,
pipetted into 1-mL vials, and then stored at −80° C. until binding
experiments were conducted. The concentration of protein was
determined using a BCA Protein Assay Kit from Pierce
Bio-technology (Rockford, III.).

| Compound ID | hA₁AR Ki | hA_{2A}AR Ki | hA₃AR Ki |
|---|---|---|---|
| T13.1 | + | +++ | ++ |
| T13.2 | + | ++ | +++ |
| T13.3 | + | +++ | +++ |
| T13.4 | ++ | ++ | +++ |
| T13.5 | +++ | +++ | +++ |
| T13.6 | ++ | ++ | +++ |
| T14.1 | ++ | ++ | +++ |
| T14.2 | +++ | ++ | +++ |
| T14.3 | +++ | ++ | + |
| T14.4 | +++ | ++ | ++ |
| T14.5 | ++ | +++ | +++ |
| T14.6 | ++ | ++ | ++ |

+ = <1 μM; ++ = 1 μM-10 μM; +++ = >10 μM

TABLE 16
Stability of prodrugs in mouse plasma after 2 hours of treatment
Assay Description:
Plasma and test compound or control compound (100 μM in DMSO) are
added to the individual wells of a 96-well microtiter plate. The
plate is incubated at 37° C. with gentle agitation for 2 hours
and quenching solution (25/50 ng/mL terfenadine/tolbutamide in
ACN/MeOH (1:1, v/v)) is added. After mixing, the quenched aliquots
are centrifuged and supernatant is withdrawn for analysis by LC-MS/MS.
The slope of the in (% remaining) vs. time point line is used to
calculate t½ according to the following formula:
Half life (t½) = −In(2)/Slope

| | Parent prodrug remained |
|---|---|
| Compound ID | after 2 hours |
| T1.1 | 11% |
| T3.1 | 14% |
| T4.1 | 21% |
| T8.1 | 9% |
| T9.8 | 17% |

[1] V. V. Gurevich, E. V. Gurevich, GPCRs and Signal Transducers: Interaction Stoichiometry, Trends in pharmacological sciences (2018).
[2] N. Wettschureck, S. Offermanns, Mammalian G proteins and their cell type specific functions, Physiological reviews 85(4) (2005) 1159-204.
[3] H. B. Schiöth, R. Fredriksson, The GRAFS classification system of G-protein coupled receptors in comparative perspective, General and comparative endocrinology 142(1-2) (2005) 94-101.
[4] G. Hasko, J. Linden, B. Cronstein, P. Pacher, Adenosine receptors: therapeutic aspects for inflammatory and immune diseases, Nature reviews. Drug discovery 7(9) (2008) 759-70.
[5] J. A. Zablocki, L. Wu, J. Shryock, L. Belardinelli, Partial A1 adenosine receptor agonists from a molecular perspective and their potential use as chronic ventricular rate control agents during atrial fibrillation (AF), Current topics in medicinal chemistry 4(8) (2004) 839-854.
[6] H. Fraser, Z. Gao, M. J. Ozeck, L. Belardinelli, N-[3-(R)-tetrahydrofuranyl]-6-aminopurine riboside, an A1 adenosine receptor agonist, antagonizes catecholamine-induced lipolysis without cardiovascular effects in awake rats, Journal of Pharmacology and Experimental Therapeutics 305(1) (2003) 225-231.
[7] M. Bayes, X. Rabasseda, J. R. Prous, Gateways to clinical trials, Methods and findings in experimental and clinical pharmacology 25(10) (2003) 831-55.
[8] V. Shneyvays, T. Zinman, A. Shainberg, Analysis of calcium responses mediated by the A3 adenosine receptor in cultured newborn rat cardiac myocytes, Cell calcium 36(5) (2004) 387-396.
[9] W. R. Tracey, W. Magee, H. Masamune, J. J. Oleynek, R. J. Hill, Selective activation of adenosine A3 receptors with N 6-(3-chlorobenzyl)-5′-N-methylcarboxamidoadenosine (CB-MECA) provides cardioprotection via KATP channel activation, Cardiovascular research 40(1) (1998) 138-145.
[10] S. Mozzicato, B. V. Joshi, K. A. Jacobson, B. T. Liang, Role of direct RhoA-phospholipase D1 interaction in mediating adenosine-induced protection from cardiac ischemia, The FASEB journal 18(2) (2004) 406-408.
[11] C. W. Schindler, M. Karcz-Kubicha, E. B. Thorndike, C. E. Müller, S. R. Tella, S. Ferré, S. R. Goldberg, Role of central and peripheral adenosine receptors in the cardiovascular responses to intraperitoneal injections of adenosine A1 and A2A subtype receptor agonists, British journal of pharmacology 144(5) (2005) 642-650.
[12] K. Varani, F. Portaluppi, S. Gessi, S. Merighi, E. Ongini, L. Belardinelli, P. A. Borea, Dose and time effects of caffeine intake on human platelet adenosine A2A receptors: functional and biochemical aspects, Circulation 102(3) (2000) 285-289.
[13] T. Maemoto, M. Tada, T. Mihara, N. Ueyama, H. Matsuoka, K. Harada, T. Yamaji, K. Shirakawa, S. Kuroda, A. Akahane, A. Iwashita, N. Matsuoka, S. Mutoh, Pharmacological characterization of FR194921, a new potent, selective, and orally active antagonist for central adenosine A1 receptors, Journal of pharmacological sciences 96(1) (2004) 42-52.
[14] J. Sawynok, Adenosine receptor activation and nociception, European journal of pharmacology 347(1) (1998) 1-11.
[15] B. Johansson, L. Halldner, T. V. Dunwiddie, S. A. Masino, W. Poelchen, L. Giménez-Llort, R. M. Escorihuela, A. Fernández-Teruel, Z. Wiesenfeld-Hallin, X.-J. Xu, Hyperalgesia, anxiety, and decreased hypoxic neuroprotection in mice lacking the adenosine A1 receptor, Proceedings of the National Academy of Sciences 98(16) (2001) 9407-9412.
[16] W.-P. Wu, J.-X. Hao, L. Halldner, C. Lövdahl, G. E. DeLander, Z. Wiesenfeld-Hallin, B. B. Fredholm, X.-J. Xu, Increased nociceptive response in mice lacking the adenosine A1 receptor, Pain 113(3) (2005) 395-404.
[17] K. Xu, E. Bastia, M. Schwarzschild, Therapeutic potential of adenosine A2A receptor antagonists in Parkinson's disease, Pharmacology & therapeutics 105(3) (2005) 267-310.
[18] R. A. Hauser, J. P. Hubble, D. D. Truong, I. U.-S. Group, Randomized trial of the adenosine A2A receptor antagonist istradefylline in advanced PD, Neurology 61(3) (2003) 297-303.
[19] J.-F. Chen, Z. Huang, J. Ma, J. Zhu, R. Moratalla, D. Standaert, M. A. Moskowitz, J. S. Fink, M. A. Schwarzschild, A2A adenosine receptor deficiency attenuates brain injury induced by transient focal ischemia in mice, Journal of Neuroscience 19(21) (1999) 9192-9200.
[20] L. Yu, Z. Huang, J. Mariani, Y. Wang, M. Moskowitz, J.-F. Chen, Selective inactivation or reconstitution of adenosine A 2A receptors in bone marrow cells reveals their significant contribution to the development of ischemic brain injury, Nature medicine 10(10) (2004) 1081.
[21] T. Olsson, T. Cronberg, A. Rytter, F. Asztély, B. B. Fredholm, M. L. Smith, T. Wieloch, Deletion of the adenosine A1 receptor gene does not alter neuronal damage following ischaemia in vivo or in vitro, European Journal of Neuroscience 20(5) (2004) 1197-1204.
[22] C. P. Turner, M. Seli, L. Ment, W. Stewart, H. Yan, B. Johansson, B. B. Fredholm, M. Blackburn, S. A. Rivkees, A1 adenosine receptors mediate hypoxia-induced ventriculomegaly, Proceedings of the National Academy of Sciences 100(20) (2003) 11718-11722.
[23] T. Porkka-Heiskanen, R. E. Strecker, M. Thakkar, A. A. Bjørkum, R. W. Greene, R. W. McCarley, Adenosine: a mediator of the sleep-inducing effects of prolonged wakefulness, Science 276(5316) (1997) 1265-1268.
[24] D. Stenberg, E. Litonius, L. Halldner, B. Johansson, B. B. Fredholm, T. Porkka-Heiskanen, Sleep and its homeostatic regulation in mice lacking the adenosine A1 receptor, Journal of sleep research 12(4) (2003) 283-290.
[25] R. Basheer, R. E. Strecker, M. M. Thakkar, R. W. McCarley, Adenosine and sleep-wake regulation, Progress in neurobiology 73(6) (2004) 379-396.
[26] S. C. Pingle, S. Mishra, A. Marcuzzi, S. G. Bhat, Y. Sekino, L. P. Rybak, V. Ramkumar, Osmotic diuretics induce adenosine A1 receptor expression and protect renal proximal tubular epithelial cells against cisplatin-mediated apoptosis, Journal of Biological Chemistry 279(41) (2004) 43157-43167.
[27] S. S. Gottlieb, D. C. Brater, I. Thomas, E. Havranek, R. Bourge, S. Goldman, F. Dyer, M. Gomez, D. Bennett, B. Ticho, BG9719 (CVT-124), an A1 adenosine receptor antagonist, protects against the decline in renal function observed with diuretic therapy, Circulation 105(11) (2002) 1348-1353.
[28] H. T. Lee, H. Xu, S. H. Nasr, J. Schnermann, C. W. Emala, A1 adenosine receptor knockout mice exhibit increased renal injury following ischemia and reperfusion, American Journal of Physiology-Renal Physiology 286(2) (2004) F298-F306.
[29] C.-X. Sun, H. W. Young, J. G. Molina, J. B. Volmer, J. Schnermann, M. R. Blackburn, A protective role for the A 1 adenosine receptor in adenosine-dependent pulmonary injury, The Journal of clinical investigation 115(1) (2005) 35-43.
[30] S. T. Holgate, The Quintiles Prize Lecture 2004: the identification of the adenosine A2B receptor as a novel therapeutic target in asthma, British journal of pharmacology 145(8) (2005) 1009-1015.
[31] J. R. Fozard, K. M. Ellis, M. F. V. Dantas, B. Tigani, L. Mazzoni, Effects of CGS 21680, a selective adenosine A2A receptor agonist, on allergic airways inflammation in the rat, European journal of pharmacology 438(3) (2002) 183-188.
[32] M. V. Sitkovsky, D. Lukashev, S. Apasov, H. Kojima, M. Koshiba, C. Caldwell, A. Ohta, M. Thiel, Physiological control of immune response and inflammatory tissue damage by hypoxia-inducible factors and adenosine A2A receptors, Annu. Rev. Immunol. 22 (2004) 657-682.
[33] A. Ohta, M. Sitkovsky, Role of G-protein-coupled adenosine receptors in downregulation of inflammation and protection from tissue damage, Nature 414(6866) (2001) 916.
[34] Q. Dong, H. Ginsberg, B. Erlanger, Overexpression of the A1 adenosine receptor in adipose tissue protects mice from obesity-related insulin resistance, Diabetes, Obesity and Metabolism 3(5) (2001) 360-366.
[35] H. Harada, O. Asano, Y. Hoshino, S. Yoshikawa, M. Matsukura, Y. Kabasawa, J. Niijima, Y. Kotake, N. Watanabe, T. Kawata, 2-Alkynyl-8-aryl-9-methyladenines as novel adenosine receptor antagonists: their synthesis and structure- activity relationships toward hepatic glucose production induced via agonism of the A2B receptor, Journal of medicinal chemistry 44(2) (2001) 170-179.
[36] L. Antonioli, C. Blandizzi, P. Pacher, G. Haskó, Immunity, inflammation and cancer: a leading role for adenosine, Nature Reviews Cancer 13(12) (2013) 842.
[37] R. H. Adamson, D. W. Zaharevitz, D. G. Johns, Enhancement of the biological activity of adenosine analogs by the adenosine deaminase inhibitor 2′-deoxycoformycin, Pharmacology 15(1) (1977) 84-89.
[38] J. Stagg, M. Smyth, Extracellular adenosine triphosphate and adenosine in cancer, Oncogene 29(39) (2010) 5346.
[39] T. Okamura, Y. Kurogi, K. Hashimoto, S. Sato, H. Nishikawa, K. Kiryu, Y. Nagao, Structure-activity relationships of adenosine A 3 receptor ligands: new potential therapy for the treatment of glaucoma, Bioorganic & medicinal chemistry letters 14(14) (2004) 3775-3779.
[40] M. Y. Avila, R. A. Stone, M. M. Civan, Knockout of A3 adenosine receptors reduces mouse intraocular pressure, Investigative ophthalmology & visual science 43(9) (2002) 3021-3026.
[41] S.-K. Kim, Z.-G. Gao, P. Van Rompaey, A. S. Gross, A. Chen, S. Van Calenbergh, K. A. Jacobson, Modeling the adenosine receptors: comparison of the binding domains of A2A agonists and antagonists, Journal of medicinal chemistry 46(23) (2003) 4847-4859.
[42] S. Tchilibon, B. V. Joshi, S.-K. Kim, H. T. Duong, Z.-G. Gao, K. A. Jacobson, (N)-Methanocarba 2, N 6-disubstituted adenine nucleosides as highly potent and selective A3 adenosine receptor agonists, Journal of medicinal chemistry 48(6) (2005) 1745-1758.
[43] S. Moro, M. Bacilieri, C. Ferrari, G. Spalluto, Autocorrelation of molecular electrostatic potential surface properties combined with partial least squares analysis as alternative attractive tool to generate ligand-based 3D-QSARs, Current drug discovery technologies 2(1) (2005) 13-21.
[44] S. Moro, Z. G. Gao, K. A. Jacobson, G. Spalluto, Progress in the pursuit of therapeutic adenosine receptor antagonists, Medicinal research reviews 26(2) (2006) 131-159.
[45] Z.-G. Gao, S.-K. Kim, T. Biadatti, W. Chen, K. Lee, D. Barak, S. G. Kim, C. R. Johnson, K. A. Jacobson, Structural determinants of A3 adenosine receptor activation: nucleoside ligands at the agonist/antagonist boundary, Journal of medicinal chemistry 45(20) (2002) 4471-4484.
[46] D. K. Tosh, A. Ciancetta, E. Warnick, S. Crane, Z.-G. Gao, K. A. Jacobson, Structure-Based Scaffold Repurposing for G Protein-Coupled Receptors: Transformation of Adenosine Derivatives into 5HT2B/5HT2C Serotonin Receptor Antagonists, Journal of medicinal chemistry 59(24) (2016) 11006-11026.
[47] S. Hess, C. E. Müller, W. Frobenius, U. Reith, K.-N. Klotz, K. Eger, 7-Deazaadenines bearing polar substituents: structure- activity relationships of new A1 and A3 adenosine receptor antagonists, Journal of medicinal chemistry 43(24) (2000) 4636-4646.

1. A compound of Formula IA or Formula IB or its pharmaceutically acceptable salt or a prodrug or metabolite thereof 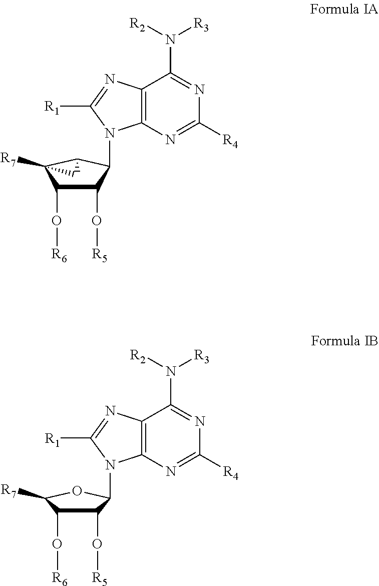
wherein
R₁ is selected from hydrogen, alkyl, alkenyl, alkynyl, aryl, heteroaryl wherein said alkyl, alkenyl, alkynyl, aryl, heteroaryl each are optionally substituted with one or more R₈;
R₂ is selected from hydrogen, amino acid, alkyl, alkenyl, alkynyl, heteroalkyl, aryl, arylalkyl, heteroaryl, heteroarylalkyl, cycloalkyl and heterocycloalkyl wherein said amino acid, alkyl, alkenyl, alkynyl, heteroalkyl, aryl, arylalkyl, heteroaryl, heteroarylalkyl, cycloalkyl and heterocycloalkyl each are optionally substituted with one or more R₈;
R₃ is selected from hydrogen, alkyl, aryl, arylalkyl, heteroaryl, heteroarylalkyl, 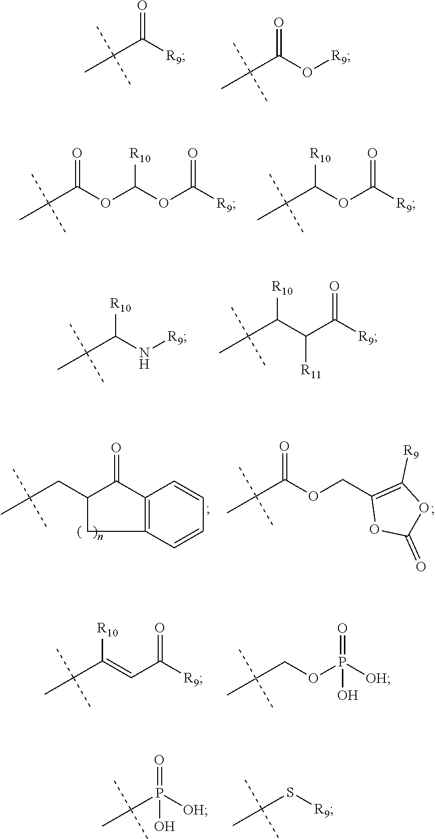
wherein n=1, 2, or 3;
R₄ is selected from hydrogen, amino acid, halogen, alkylthio, alkyl, alkenyl, alkynyl, heteroalkyl, aryl, arylalkyl, heteroaryl, heteroarylalkyl, cycloalkyl and heterocycloalkyl wherein said amino acid, alkyl, alkenyl, alkynyl, heteroalkyl, aryl, arylalkyl, heteroaryl, heteroarylalkyl, cycloalkyl and heterocycloalkyl each are optionally substituted with one or more R₈;
R₅ and R₆ are independently selected from hydrogen, alkyl, 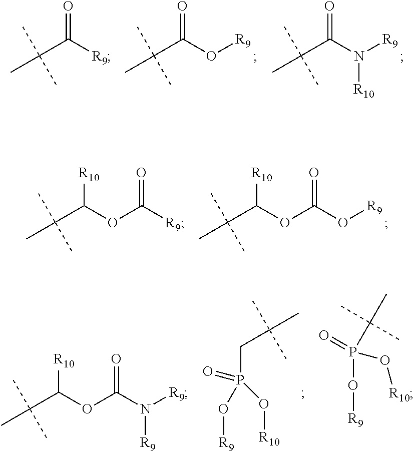
and R₅ and R₆ can be connected to form any of the following cyclic ring systems 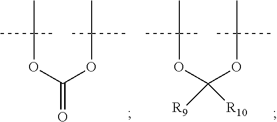
wherein R₉ and R₁₀ are selected from hydrogen, alkyl, heteroalkyl, aryl, arylalkyl, heteroaryl, heteroarylalkyl, cycloalkyl and heterocycloalkyl wherein said each are optionally substituted with one or more R₈;
and R₉ and R₁₀ can be connected to form a cycloalkyl or heterocycloalkyl include one or more nitrogen (N), oxygen (O) or sulfur (S) atoms; wherein said cycloalkyl, heterocycloalkyl are optionally substituted with one or more R₈;
R₇ is selected from hydrogen, alkyl, 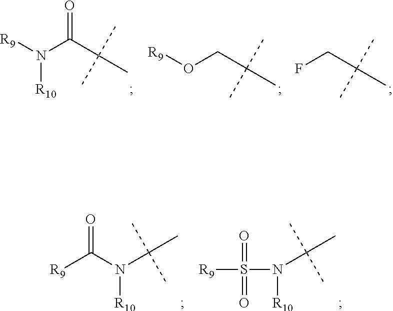
R₈ is independently selected for each occurrence from the group consisting of hydrogen, amino acid, lower alkyl, lower alkenyl, lower alkynyl, cycloalkyl, heterocycloalkyl aryl, heteroaryl, alicyclic, arylalkyl, heteroarylalkyl, lower alkoxy, aryloxy, amino, alkylamino, dialkylamino, diarylalkylamino, alkylthio, arylthio, heteroarylthio, oxo, oxa, —C(O)—R₁₂, —C(O)OR₁₂, —C(O)NR₁₂R₁₃, —C(S)NR₁₂R₁₃, CO₂H, acyloxy, halo, —CN, —NO₂, —N₃, —SH, —OH, —CH(CF₃)NR₁₂R₁₃, —C(═N)NR₁₂, perhaloalkyl, perhaloalkoxy, perhaloacyl, guanidinyl, pyridinyl, thiophene, furanyl, indolyl, indazolyl, phosphonate, phosphonic acid, phosphate, phosphoramide, sulfonate, sulfone, sulfate, sulphonamide, carbamate, urea, thiourea, thioamide and thioalkyl; wherein said heterocycloalkyl or heteroaryl include one or more nitrogen (N), oxygen (O) or sulfur (S) atoms; wherein R₁₂ and R₁₃ are independently selected from H, methyl, ethyl, and benzyl;
R₉, R₁₀, and R₁₁ are each independently selected from hydrogen, amino acid, lower alkyl, lower alkenyl, lower alkynyl, cycloalkyl, heterocycloalkyl aryl, heteroaryl, alicyclic, arylalkyl, heteroarylalkyl, lower alkoxy, aryloxy, —NO₂, —N₃, —SH, or —OH, each are optionally substituted with one or more R₈.

2. A compound of Formula II or its pharmaceutically acceptable salt or prodrug or metabolite thereof 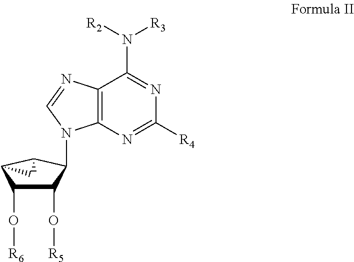
wherein R₂, R₃, R₄, R₅, and R₆ are as defined in claim 1.

3. A compound of Formula IV or its pharmaceutically acceptable salt or a prodrug or metabolite thereof 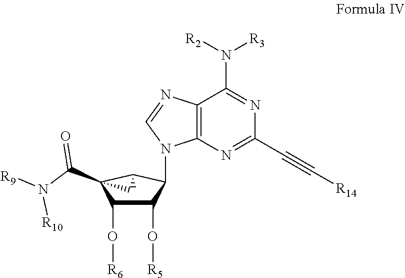
wherein R₁₄ is selected from aryl or heteroaryl which is optionally substituted with one or more R₈; and R₂, R₃, R₅, R₆, R₈, R₉, and R₁₀ are as defined in claim 1.

4. A compound of Formula VII or its pharmaceutically acceptable salt or a prodrug or metabolite thereof 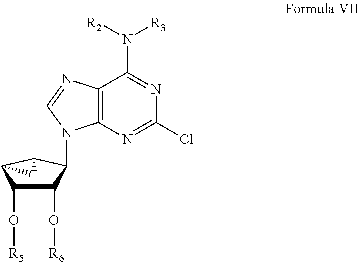
wherein R₂, R₃, R₅, and R₆ are as defined in claim 1.

5. A compound of Tables 1-14 or its pharmaceutically acceptable salt or a prodrug or metabolite thereof.

6. A compound of Table 14 or its pharmaceutically acceptable salt or a prodrug or metabolite thereof.

7. A pharmaceutical composition comprising a compound of Formula IA or Formula IB as defined in claim 1 or a pharmaceutically acceptable salt, ester or prodrug or metabolite thereof in association with a pharmaceutically acceptable diluent or carrier to form a formulation system for delivering the compound.

8. A pharmaceutical composition comprising a compound of Formula IA or Formula IB as defined in claim 1 or a pharmaceutically acceptable salt, ester or prodrug or metabolite thereof in a combination of other pharmaceutically active agent(s).

9. A compound of Formula IA or Formula IB as defined in claim 1 or a pharmaceutically acceptable salt, ester or prodrug or metabolite thereof for use in therapy.

10. The use of a compound of Formula IA or Formula IB as defined in claim 1 or a pharmaceutically acceptable salt, ester or prodrug or metabolite thereof in the preparation of a medicament for the treatment of cardiovascular disease, nervous system disorders, Parkinson's disease, pain, neurodegenerative disorders, ischaemia and neuroprotection, sleep, renal system disorders, pulmonary disorders, inflammatory disorders, endocrine disorders, cancer, and visual disorders.

11. A process of producing a compound of Formula IA or Formula IB as defined in claim 1 or its pharmaceutically acceptable salt or prodrug or metabolite.

12. A process of producing Compound A as outlined in Scheme K and the procedures describing for Compound A.

13. A modified-release formulation comprising compound of Formula IA and Formula IB a single active pharmaceutical ingredient, (a) at least one release controlling polymer selected from the group consisting of pH-dependent polymers and non-pH-dependent polymers, and (b) at least one stabilizer selected from the group consisting of acidifying agents and hydrophobizing agents, wherein the total amount of water content in the formulation is not more than 5% by weight of the formulation.

14. A modified-release formulation comprising compound A or Compound B a single active pharmaceutical ingredient, (a) at least one release controlling polymer selected from the group consisting of pH-dependent polymers and non-pH-dependent polymers, and (b) at least one stabilizer selected from the group consisting of acidifying agents and hydrophobizing agents.

15. A modified-release formulation is comprising compound A or compound B, a single active pharmaceutical ingredient, in an enteric-coated capsules at least one stabilizer selected from the group consisting of acidifying agents and hydrophobizing agents.
